(19) United States
(12) Patent Application Publication
(10) Pub. No.: US 2023/0043824 A1
(43) Pub. Date: Feb. 9, 2023

(54) ACTIVE COMPRESSION-DECOMPRESSION DEVICE INTEGRATION

(71) Applicant: ZOLL Medical Corporation, Chelmsford, MA (US)

(72) Inventor: Gary A Freeman, Waltham, MA (US)

(21) Appl. No.: 17/760,106

(22) PCT Filed: Feb. 4, 2021
(86) PCT No.: PCT/US2021/070127

§ 371 Date: Aug. 4, 2022

Related U.S. Application Data
(60) Provisional application No. 62/969,815, filed on Feb. 4, 2020.

(51) Int. Cl. A61H 31/00; A61N 1/39

(52) U.S. Cl. CPC A61H 31/005; A61H 31/007; A61N 1/39044; A61H 2031/001; A61H 2201/0153; A61H 2201/5061; A61H 2201/5084; A61H 2201/5043; A61H 2201/5097

(57) ABSTRACT

A system is provided for resuscitative therapy to a patient by delivering active chest compression decompressions. The system may include an ACD device configured to be coupled to the patient's chest and constructed for a rescuer to press and pull on the ACD device to administer active compression decompression therapy. Additionally, the ACD device may include at least one sensor for sensing at least one active compression decompression parameter, processing circuitry configured to process the at least one active compression decompression parameter and provide an output based on the at least one parameter, a first communication circuit configured to transfer data related to the processed output of the at least one parameter, and a second communication circuit capable of being removably coupled to either the electrode assembly or the ACD device.

### BACKGROUND

Survival rates for patients of cardiac arrest are significantly reduced for every minute of delay in providing resuscitative care. Therefore, the efficacy of the resuscitative care depends on a timely initiation of care with minimal delays and/or interruptions during administration of care. Cardiopulmonary resuscitation (CPR), which includes chest compressions and ventilations, is one form of resuscitative care provided in response to a cardiac arrest. A patient monitor (e.g., automated external defibrillator or AED or a professional defibrillator) may also be used in conjunction with CPR.

A first responder to a rescue event (e.g., cardiac arrest) may initiate care on a patient (or victim) using resuscitation equipment such as an external defibrillator and/or a chest compression device that provides instruction, coaching, and/or mechanical assistance in the delivery of CPR. The first responder may transfer care of the cardiac arrest patient to a second responder that has different or possibly more advanced equipment. For example, a bystander may initiate care and transfer the patient to an ambulance squad. As another example, an ambulance squad may initiate care and transfer the patient to an emergency room team. During these types of transfers, the second responders may utilize additional and/or more advanced resuscitation equipment. The quality of the resuscitation effort may depend on the efficiency of this equipment transition. Additionally, an efficient transfer of any data collected during the initial response effort to the subsequent equipment and/or personnel may further improve the medical response and patient outcome.

Examples of resuscitative care equipment may include a chest compression device such as an active compression-decompression (ACD) device, which may significantly enhance blood circulation due to increased loading of blood into the heart and ejection of blood out to the other organs. Chest compressions during CPR help maintain blood, and consequently oxygen, circulation through the body, heart, and brain, which are the organs that can sustain substantial damage from an adverse cardiac event. Traditional chest compressions include two phases. During the first phase, which may be referred to as an active compression phase, a rescuer applies external pressure to compress (i.e., push downward) the chest, resulting in blood flow from the heart to peripheral tissues. During the second phase, which may be referred to as a relaxation phase, the rescuer releases the external pressure, allowing for venous return of blood back to the heart. Due to the natural elasticity of the chest wall, in response to the release of external pressure, the chest expands back to its original position prior to the compression. The expansion of the chest enables the cardiac chambers to at least partially refill with blood that is then available to circulate in response to the next active compression. An ACD device is configured to pull up on the chest to actively decompress (i.e., pull upward) the chest of the patient. This active decompression provides a greater expansion of the chest than that provided by the natural elasticity of the chest wall. This greater expansion of the chest results in a reduction in intrathoracic pressure, which may improve venous refilling of the heart (i.e., enables larger volume of blood to fill the heart), to enhance the circulation provided by the chest compressions.

### SUMMARY

Herein, an active compression decompression parameter can include any parameter associated with chest compressions or decompressions, for example.

An example, according to some embodiments of the disclosure, of a system for providing resuscitative therapy to a patient by delivering active chest compression decompressions, includes: an ACD device configured to be coupled to the patient's chest and constructed for a rescuer to press and pull on the ACD device to administer active compression decompression therapy, the ACD device including: at least one sensor configured for sensing at least one active compression decompression parameter, processing circuitry configured to process the at least one active compression decompression parameter and generate output based at least in part on the at least one parameter, and a first communication circuit configured for use in transferring data related to the output from the ACD device to a patient monitor; and a second communication circuit, configured for use in the transferring of the data related to the output from the ACD device to the patient monitor, capable of being removably coupled to at least one of the ACD device and the patient monitor.

Some embodiments my include one or more of the following features. The system includes a connector, including the second communication circuit, for use in establishing communication from the ACD device to the patient monitor. The at least one sensor includes at least one of a motion sensor, an accelerometer, and a force sensor. The motion sensor is configured to monitor a motion of at least a portion of the patient's chest in response to force applied during the active compression decompression therapy. The force sensor is configured to monitor force applied to at least a portion of the patient's chest during the active compression decompression therapy. The ACD device includes a handle, wherein the at least one sensor is located within the handle. The ACD device includes a shaft, wherein the at least one sensor is located within the shaft. The ACD device includes a housing, wherein the first communication circuit is disposed within the housing. The ACD device includes a pad configured to be adhered to and to cover at least a portion of the patient's chest. The ACD device includes a shaft configured to be removably coupled to the pad. The pad includes a mount configured to receive the shaft. The at least one sensor is located within the pad. The pad includes at least one receptacle for receiving an electrode assembly of the patient monitor. The at least one receptacle includes a first recess having a first shape complementary to a first electrode of the electrode assembly, and a second recess having a second shape complementary to a second electrode of the electrode assembly. The pad includes indicia for guiding placement of the electrode assembly. The electrode assembly includes a first electrode and a second electrode electrically coupled to one another and each configured to be adhered to the patient to deliver electrical therapy to the patient.

Furthermore, in some embodiments, the second communication circuit is capable of being removably coupled to the ACD device. The patient monitor includes a defibrillator. The electrode assembly includes a sensor configured to provide data related to chest compressions. The second communication circuit is configured to transfer data related to chest compressions when the connector is coupled to the patient monitor. The second communication circuit is configured to transfer data related to chest compressions when the connector is coupled to the ACD device. The second communication circuit is capable of being removably coupled to the patient monitor. The second communication circuit couples with a sensor of the patient monitor. The sensor of the patient monitor is a motion sensor. A sensor of the patient monitor is an accelerometer. The first and second communication circuits are configured to mutually establish a wireless communications channel. The wireless communications channel is a near field wireless communications channel. The wireless communications channel is a far field wireless communications channel. The first and second communication circuits include at least one of an RF chip, a NFC chip, a Bluetooth chip. The connector includes connecting components including at least one of detents, fasteners, magnets, locking features and one or more cables. The connector includes a receptacle provided with the ACD device and in electrical communication with the first communication circuit, and a complementary connector is provided with the patient monitor. The connector is provided with the ACD device and in electrical communication with the first communication circuit, and a complementary receptacle is provided with the patient monitor. The connector includes at least one magnetic coupling component. The at least one magnetic coupling component includes a first magnetic component provided with the ACD device and a second magnetic component provided with the patient monitor. The first and second communication circuits are configured to establish communication upon coupling of the first and second magnetic components. The connector includes a removably connectable memory card. A user interface includes one or more controls, the user interface being configured to display one or more of cardio-pulmonary resuscitation (CPR) instructions, defibrillation instructions and feedback information related to treatment of the patient.

An example, according to some embodiments of the disclosure, of an apparatus for providing resuscitative therapy to a patient by delivering active chest compression decompressions, includes: an active chest compression decompression (ACD) device configured to be coupled to the patient's chest and constructed for a rescuer to press and pull on the ACD device to administer active compression decompression therapy, the ACD device including: at least one sensor for sensing at least one active compression decompression parameter, processing circuitry configured to process the at least one active compression decompression parameter and generate an output based on the at least one parameter, and a first communication circuit configured to transfer data related to the output from the ACD device to a patient monitor; and a connector configured for use in establishing communication from the first communication circuit to a second communication circuit of the patient monitor.

Some embodiments my include one or more of the following features. The at least one sensor includes at least one of a motion sensor, an accelerometer and a force sensor. The ACD device includes a handle, and wherein the at least one sensor is located within the handle. The ACD device includes a shaft, and the at least one sensor is located within the shaft. The ACD device includes a pad configured to be adhered to and to cover at least a portion of the patient's chest. The ACD device includes a shaft configured to be removably coupled to the pad. The pad includes a mounting plate configured to receive the shaft. The at least one sensor is located within the pad. The pad includes at least one receptacle for receiving an electrode assembly of the patient monitor. The at least one receptacle includes a first recess having a first shape complementary to a first electrode of the electrode assembly, and a second recess having a second shape complementary to a second electrode of the electrode assembly. The pad includes indicia for guiding placement of the electrode assembly. The ACD device includes a housing, and wherein the first communication circuit is disposed within the housing of the ACD device. The first and second communication circuits are configured to mutually establish a wireless communications channel. The connector includes a receptacle configured to receive a complementary connector provided with the electrode assembly, for establishing the communication between the corresponding communication circuits. The connector includes a connector configured to receive a complementary receptacle provided with the electrode assembly, for establishing the communication. The connector includes at least one magnetic coupling component.

An example, according to some embodiments of the disclosure, of an apparatus for providing resuscitative therapy to a patient, includes: an electrode assembly configured to deliver electrical therapy to the patient in cardiac arrest; a first communication circuit configured to be removably coupled to the electrode assembly, and to transfer data related to chest compressions; and a connector, including the first communication circuit, configured for use in establishing communication between the first communication circuit and a second communication circuit of an active chest compression decompression (ACD) device for providing ACD therapy.

Some embodiments my include one or more of the following features. The electrode assembly includes a first electrode and a second electrode electrically coupled to one another and each configured to be adhered to the patient to deliver the electrical therapy to the patient. The system includes a defibrillator connected to or including the electrode assembly, and wherein the transfer of the data related to chest compressions is from the ACD device to the defibrillator. The electrode assembly includes a sensor configured to provide data related to chest compressions. The first communication circuit is configured to transfer data related to chest compressions to the defibrillator when the connector is coupled to the electrode assembly. The first communication circuit is configured to transfer the data related to the chest compressions when the connector is coupled to the electrode assembly. The first communication circuit is removably coupled to the sensor. The first communication circuit includes a sensor. The sensor is a motion sensor. The sensor is an accelerometer. The connector includes a receptacle configured to receive a complementary connector provided with the ACD device, for establishing the communication. The connector includes a connector configured to receive a complementary receptacle provided with the ACD device, for establishing the communication. The connector includes at least one magnetic coupling component. The connector includes a removably connectable memory card.

An example, according to some embodiments of the disclosure, of a system for providing resuscitative therapy to a patient by delivering active chest compression decompressions and electrotherapy, includes: an active chest compression decompression (ACD) device configured to be coupled to the patient's chest and constructed for a rescuer to press and pull on the ACD device to administer ACD therapy, the ACD device including: at least one sensor for sensing at least one active compression decompression parameter, processing circuitry configured to process the at least one active compression decompression parameter and generate output based on the at least one parameter, and a first communication circuit configured for use in transferring data related to the output; and a second communication circuit provided with a defibrillator system and configured for use in transferring the data from the ACD device to the defibrillator system.

Some embodiments my include one or more of the following features. The at least one sensor includes at least one of a motion sensor, an accelerometer and a force sensor. The ACD device includes a handle, and wherein the at least one sensor is located within the handle. The ACD device includes a shaft, and wherein the at least one sensor is located within the shaft. The ACD device includes a pad configured to be adhered to and to cover at least a portion of the patient's chest. The ACD device includes a shaft configured to be removably coupled to the pad. The pad includes a mounting plate configured to receive the shaft. The at least one sensor is located within the pad. The pad includes at least one receptacle for receiving an electrode assembly of the defibrillator system. The at least one receptacle includes a first recess having a first shape complementary to a first electrode of the electrode assembly, and a second recess having a second shape complementary to a second electrode of the electrode assembly. The ACD device includes a housing, and wherein the first communication circuit is disposed within the housing. The defibrillator system includes a defibrillator housing, at least one cable and an electrode assembly. The second communication circuit is provided within the defibrillator housing. The second communication circuit is provided with the at least one cable. The second communication circuit is provided with the electrode assembly. The first and second communication circuits are configured to establish a wireless communications channel between the ACD device and the defibrillator system. The wireless communications channel is a near field wireless communications channel. The wireless communications channel is a far field wireless communications channel. The first and second communication circuits include at least one of an RF chip, a NFC chip, a Bluetooth chip.

An example, according to some embodiments of the disclosure, of a system for providing resuscitative therapy to a patient, includes: an ACD device configured to be coupled to the patient's chest and constructed for a rescuer to press and pull on the ACD device to administer active compression decompression therapy; a receptacle configured to enable docking and establishment of an electrical communication between the ACD device and a mobile computing device having at least one sensor; and one or more processors configured to receive and analyze signals from the at least one sensor of the mobile computing device for assisting resuscitative therapy upon detecting that the electrical communication is established.

Some embodiments my include one or more of the following features. The mobile computing device includes at least one of a tablet and a phone. The system includes a user interface for providing input to the one or more processors. The at least one sensor includes a motion sensor, a camera and an impedance sensor. The receptacle is disposed on the ACD device. The receptacle includes a coupling region complementary to a corresponding coupling region of the ACD device.

An example, according to some embodiments of the disclosure, of a method of providing resuscitative therapy to a patient by delivering active chest compression decompressions, includes: adhering a multifunctional landing pad to a chest of the patient, the multifunctional landing pad including a motion sensor for sensing chest wall motion associated with compressions applied to the patient and at least one mechanical coupling feature for mounting an active compression decompression (ACD) device to the multifunctional landing pad; applying manual chest compressions to the patient via the multifunctional landing pad; at least temporarily stopping the manual chest compressions; engaging the at least one mechanical coupling feature of the multifunctional landing pad with a complementary at least one mechanical coupling feature of the ACD device to couple the ACD device to the multifunctional landing pad; establishing communication between the ACD device and a patient monitor for transmission of chest compression data from the ACD device to the patient monitor; and applying ACD treatment to the patient via the ACD device.

Some embodiments my include one or more of the following features. The method includes including applying the ACD treatment, wherein the patient monitor includes a defibrillator. The method includes applying the ACD treatment, wherein the defibrillator is coupled to or includes one or more electrodes positioned on the patient. The method includes applying the ACD treatment, wherein the one or more electrodes are positioned on the patient without interruption during the providing of the resuscitative therapy to a patient. The establishing of the communication includes coupling of a connector, attached to or part of the defibrillator, to at least one of the ACD device, the multifunctional landing pad, or the patient monitor. The establishing of the communication includes the ACD device and the defibrillator wirelessly coupling with each other.

An example, according to some embodiments of the disclosure, of a method of providing resuscitative therapy to a patient by delivering active chest compression decompressions, includes: adhering an ACD device to the chest of the patient via an ACD device landing pad, the ACD device including a motion sensor for sensing chest wall motion associated with ACD treatment applied to the patient and a force sensor for sensing force associated with ACD treatment applied to the patient; applying ACD treatment to the patient via the ACD device; without removing the ACD device from the chest of the patient, applying an electrode assembly, of or coupled to a patient monitor, to the patient at one or more locations adjacent to the ACD device landing pad; and establishing communication between the ACD device and the patient monitor, for transmission of chest compression data from the ACD device to the patient monitor.

Some embodiments my include one or more of the following features. The ACD device includes the ACD device landing pad, and wherein the adhering of the ACD device to the chest of the patient via the ACD device landing pad includes adhering the ACD device to the chest of the patient via the ACD device landing pad of the ACD device. The method includes adhering the ACD device landing pad to the chest of the patient prior to the adhering of the ACD device to the chest of the patient via the ACD device landing pad, and wherein the adhering of the ACD device to the chest of the patient via the ACD landing pad includes coupling the ACD device to the ACD device landing pad after the adhering of the ACD device landing pad to the chest of the patient. The method includes applying the ACD treatment, wherein the patient monitor includes a defibrillator. The method includes applying the ACD treatment, wherein the defibrillator is coupled to or includes the electrode assembly. The establishing of the communication includes coupling a connector to at least one of the ACD device and the defibrillator to connect the ACD device to the defibrillator. The establishing of the communication includes the ACD device and the defibrillator wirelessly coupling with each other.

An example, according to some embodiments of the disclosure, of a system for providing resuscitative therapy to a patient by delivering active chest compression decompressions, includes: an active compression decompression (ACD) device configured to be coupled to a patient's chest and configured such that a rescuer can press and pull on the ACD device to administer active compression decompression therapy, the ACD device including: at least one sensor configured to sense at least one active compression decompression parameter, and at least one processor and at least one memory, the at least one processor being configured to process the at least one parameter and generate output based at least in part on the at least one parameter; and a connector, including a first communication circuit, configured to be plugged into at least one of the ACD device and a patient monitor, wherein the connector is configured for use in connecting the ACD device with the patient monitor to enable transfer of the output from the ACD device to the patient monitor.

Some embodiments my include one or more of the following features. The patient monitor includes a defibrillator including or attached to at least one electrode, wherein the at least one electrode is configured to be positioned on the patient. The connector is configured such that the ACD device can be positioned on the patient's chest, to enable delivery of ACD treatment, and connected with the defibrillator while the at least one electrode is positioned on the patient, to enable delivery of electrotherapy, and without requiring removal of the at least one electrode from the patient. The connector is configured such that the at least one electrode can be positioned on the patient, to enable delivery of electrotherapy, and connected with the ACD device while the ACD device is positioned on the patient's chest, to enable delivery of ACD treatment, without requiring removal of the ACD device from the patient's chest. The patient monitor includes a second communication circuit for use in connecting the ACD device with the patient monitor to enable the transfer of the output from the ACD device to the patient monitor. The ACD device includes a landing pad. The ACD device is configured to couple with a landing pad positioned on the patient's chest.

An example, according to some embodiments of the disclosure, of a system for providing resuscitative therapy to a patient by delivering active chest compression decompressions, includes: an active compression decompression (ACD) device configured to be positioned on patient's chest and configured such that a rescuer can press and pull on the ACD device to administer active compression decompression therapy, the ACD device including: at least one sensor configured to sense at least one active compression decompression parameter, and at least one processor and at least one memory, the at least one processor being configured to process the at least one parameter and generate output based at least in part on the at least one parameter; and a connector, including a first communication circuit, configured to be plugged into at least one of the ACD device or a defibrillator, while at least one electrode of the defibrillator is positioned on the patient, to enable delivery of electrotherapy to the patient, wherein the connector is configured for use in connecting the ACD device with the defibrillator to enable transfer of the output from the ACD device to the defibrillator.

Some embodiments my include one or more of the following features. The system includes a patient monitor, wherein the patient monitor includes the defibrillator. The connector is configured such that the at least one electrode can be positioned on the patient, and the patient monitor can be connected with the ACD device, without requiring removal of the ACD device from the patient's chest. The patient monitor includes a second communication circuit for use in connecting the ACD device with the patient monitor to enable the transfer of the output from the ACD device to the patient monitor. The ACD device includes a landing pad. The ACD device is configured to couple with a landing pad positioned on the patient's chest.

An example, according to some embodiments of the disclosure, of a system for providing resuscitative therapy to a patient by delivering active chest compression decompressions, includes: an active compression decompression (ACD) device configured to be positioned on a patient's chest and configured such that a rescuer can press and pull on the ACD device to administer active compression decompression therapy, the ACD device including: at least one sensor configured to sense at least one active compression decompression parameter, and at least one processor and at least one memory, the at least one processor being configured to process the at least one parameter and generate an output based at least in part on the at least one parameter; wherein the ACD device is configured such that: while the ACD device is positioned on the patient's chest, at least one electrode, connected to or part of a defibrillator, can be positioned on the patient, to enable delivery of electrotherapy, without removing the ACD device from the patient's chest, and while the ACD device is positioned on the patient's chest, when the defibrillator comes within sufficient proximity with the ACD device, the ACD device and the defibrillator wirelessly couple such that the output can be wirelessly transmitted from the ACD device to the defibrillator.

Some embodiments my include one or more of the following features. The ACD device includes a first communication circuit and the defibrillator includes a second communication circuit, and wherein the first communication circuit and the second communication circuit are configured for use in enabling the ACD device and the defibrillator to wirelessly couple. The system includes a patient monitor, wherein the patient monitor includes the defibrillator. The system is configured such that the at least one electrode can be positioned on the patient, to enable delivery of electrotherapy, without requiring removal of the ACD device from the patient's chest. The ACD device includes a landing pad. The ACD device is configured to couple with a landing pad positioned on the patient's chest.

An example, according to some embodiments of the disclosure, of a system for providing resuscitative therapy to a patient by delivering active chest compression decompressions, includes: an active compression decompression (ACD) device configured to be positioned on a patient's chest and configured such that a rescuer can press and pull on the ACD device to administer active compression decompression therapy, the ACD device including: at least one sensor configured to sense at least one active compression decompression parameter, and at least one processor and at least one memory, the at least one processor being configured to process the at least one parameter and generate an output based at least in part on the at least one parameter; wherein the ACD device is configured such that: while at least one electrode of a defibrillator is positioned on the patient, to enable delivery of electrotherapy, the ACD device can be positioned on the chest of the patient without removing the at least one electrode from the patient, and while the at least one electrode is positioned on the patient, to enable delivery of electrotherapy, when the ACD device comes within sufficient proximity with the defibrillator, the defibrillator and the ACD device wirelessly couple such that the output can be wirelessly transmitted from the ACD device to the defibrillator.

Some embodiments my include one or more of the following features. The ACD device includes a first communication circuit and the defibrillator includes a second communication circuit, and wherein the first communication circuit and the second communication circuit are configured for use in enabling the ACD device and the defibrillator to wirelessly couple. The system includes a patient monitor, wherein the patient monitor the defibrillator. The system is configured such that the ACD device can be positioned on the patient's chest, to enable delivery of active chest compression decompressions, without requiring removal of the at least one electrode from the patient. The ACD device includes a landing pad. The ACD device is configured to couple with a landing pad positioned on the patient's chest.

An example, according to some embodiments of the disclosure, of a method of providing resuscitative therapy to a patient by delivering active chest compression decompressions, includes: applying manual chest compressions to the patient; at least temporarily stopping the manual chest compressions; positioning an active compression decompression (ACD) device on the patient's chest; establishing communication between the ACD device and a patient monitor, for transmission of chest compression data between the ACD device and the patient monitor; and applying ACD treatment to the patient via the ACD device.

Some embodiments my include one or more of the following features. The method includes applying the ACD treatment, wherein the patient monitor includes a defibrillator. The method includes applying the ACD treatment, wherein the defibrillator is coupled to or includes one or more electrodes positioned on the patient. The method includes applying the ACD treatment, wherein the one or more electrodes are positioned on the patient without interruption during the providing of the resuscitative therapy to the patient. The establishing of the communication includes coupling of a connector to the ACD device or a landing pad for the ACD device. The establishing of the communication includes coupling of a connector to the patient monitor. The establishing of the communication includes the ACD device and the defibrillator wirelessly coupling with each other. The ACD device includes a landing pad, and wherein positioning the ACD device on the patient's chest includes positioning the landing pad of the ACD device on the patient's chest.

Some embodiments my include one or more of the following features. The method includes positioning a landing pad for the ACD device on the patient's chest prior to the positioning of the ACD device on the patient's chest, wherein the positioning of the ACD device on the patient's chest includes coupling the ACD device to the landing pad.

### BRIEF DESCRIPTION OF THE DRAWINGS

Various aspects of the disclosure are discussed below with reference to the accompanying figures, which are not intended to be drawn to scale. The figures are included to provide an illustration and a further understanding of various examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure. A quantity of each component in a particular figure is an example only and other quantities of each, or any, component could be used.

FIG. 1A is a schematic diagram of a rescue event in which a rescuer provides manual chest compressions to a patient while utilizing an electrode assembly.

FIG. 1B is a schematic diagram of a rescue event in which the rescuer is using an active compression-decompression (ACD) device applied directly to the patient to provide active compression decompression therapy.

FIG. 2 is a schematic diagram of an ACD device used with an ACD device landing pad and an ACD device mount in accordance with an embodiment.

FIG. 3A is a block diagram illustrating components of an active compression-decompression (ACD) device in wireless communication with an ACD device landing pad in accordance with an embodiment.

FIG. 3B is a block diagram illustrating an alternative embodiment showing an active compression-decompression (ACD) device having an electrical connection with the ACD device landing pad.

FIGS. 4A-4B are block diagrams illustrating various configurations for affixing the ACD device to an ACD device landing pad in accordance with various embodiments.

FIGS. 4C-4E are block diagrams illustrating an embodiment for affixing the ACD device to a multifunctional compression pad.

FIG. 5A is schematic diagram illustrating an interface displayed on a handle of an ACD device in accordance with an embodiment.

FIG. 5B is schematic diagram illustrating an alternative embodiment of the handle of an ACD device, which may include a mobile computing device mount.

FIG. 6A is a flow chart illustrating steps performed by rescuers during a rescue event in which rescuers swap a manual chest compression pad for the ACD device landing pad in accordance with an embodiment.

FIG. 6B is a flow chart illustrating the steps performed by rescuers during a rescue event in which rescuers utilize a multifunctional chest compression pad in accordance with an embodiment.

FIGS. 7A-7D are schematic diagrams of a rescue system and illustrate an example of how to swap a manual compression pad for an ACD device landing pad during a rescue event in which electrodes of a patient monitor have already been placed on the patient.

FIGS. 8A and 8B are schematic diagrams of alternative configurations of a rescue system described with respect to FIGS. 7A-7D in accordance with various embodiments.

FIG. 8C is a schematic diagram illustrating a rescue system that may include shaped corners to promote proper integration and orientation of components of the rescue system in accordance with an embodiment.

FIG. 9 is a flow chart illustrating the steps performed by rescuers during a rescue event in accordance with an embodiment.

FIGS. 10A-10C are schematic diagrams of the rescue system and illustrate an example of how to place electrodes onto a patient during a rescue event in which the ACD device has already been placed and is in use.

FIG. 10D is a schematic diagram of an alternative embodiment of FIGS. 10A-10C that may include wireless communication between the patient monitor and ACD device.

FIGS. 11A-11E are block diagrams of the rescue system illustrating different rescue system configurations in accordance with various embodiments.

FIG. 12 is a schematic diagram of a patient monitor.

### DETAILED DESCRIPTION

In an emergency resuscitative effort (e.g., cardiac arrest), the patient is in need of immediate care, however, it is often the case that the appropriate equipment for providing such care is not readily available, arriving at a later time. What is more, the relevant resuscitation equipment may arrive on the emergency scene at different times. For instance, a bystander may witness a person suffering from cardiac arrest and immediately begin to perform manual CPR chest compressions, without any equipment available. Another bystander may retrieve a nearby publicly accessible AED (e.g., from a wall cabinet) and bring the device to the scene, to apply the electrodes of the AED to the victim. A more advanced life support crew may subsequently arrive, with more advanced equipment for treating the patient, such as an ACD device and a more advanced defibrillator with patient monitoring capabilities. As it may be preferable to use the more advanced life support equipment, e.g., ACD device in place of standard manual chest compressions with the hands, or advanced defibrillator/monitor in place of a publicly accessible AED, it may be undesirable to take the time to switch out the equipment when life-giving CPR could be given to the patient. In addition, when a transition of equipment takes place, it would be advantageous to have any data or records generated by the initial (basic) set of equipment available for further processing or consideration by a subsequent (more advanced) set of equipment. Hence, in accordance with embodiments described herein, it may be more favorable to be able to provide a system that allows for staged integration of life-saving equipment over the course of a resuscitation effort in an intuitive and efficient manner.

Techniques and systems are presented herein for enabling efficient and seamless transitions and/or integration of equipment used in various stages of a resuscitative care in response to a cardiac arrest and for continuity of data collection during these various stages. The resuscitative care response may involve the use of various specialized equipment such as an active compression-decompression (ACD) device, an ACD device landing pad (or multifunctional chest compression pad), and a patient monitor (e.g., an external defibrillator with patient monitoring capabilities), which may arrive to the emergency scene at different times. In various embodiments, an ACD device may or may not include a landing pad. In some embodiments, an ACD device including a landing pad may be positioned on the chest of a patient via the landing pad of the ACD device. In some embodiments, a landing pad may be positioned on the chest of a patient, and then an ACD device may be coupled to the landing pad. In various embodiments, a landing pad can be or include, e.g., an ACD landing pad, a multifunctional landing pad, or another type of landing pad.

As one example, a first responder may initiate resuscitative care for a patient of the cardiac arrest by providing chest compressions with an ACD device. A second responder may arrive subsequently with an external defibrillator and continue resuscitative care by monitoring the patient (e.g., monitoring ECG, end tidal carbon dioxide, pulse oximetry, blood pressure, etc.), applying electrode pads and using the external defibrillator to analyze the patient's heart rhythm and determine whether defibrillation and/or other care supported by the external defibrillator (e.g., pacing) is necessary.

The ACD device may include one or more sensors (e.g., motions sensors, accelerometers, force/pressure sensors). The one or more sensors may generate signals indicative of chest compression motion on downstroke and upstroke. The signals are received by at least one processor and analyzed, filtered, or further processed to generate chest compression/decompression data, stored by the ACD device and/or another computing device separate from the ACD device. This data may be useful to assist in determining various aspects of the resuscitation, for example, rescuer feedback, defibrillation timing, and ventilation timing, to list a few examples. Additionally, or alternatively, it may be necessary and/or more convenient or efficient for the external defibrillator/monitor to collect, analyze, and/or display the motion sensor data in lieu of or in addition to such collection analysis, and/or display by the ACD device.

Hence, in accordance with various aspects of the present disclosure, it is desirable to provide a connection interface that may quickly and easily establish a connection between the external defibrillator/monitor and the ACD device. Further it is desirable for such an apparatus to provide data communication between the ACD device and the external defibrillator, for example, enabling the external defibrillator/monitor to have access to data obtained by the ACD device prior to when the external defibrillator/monitor had arrived. In other words, in a situation where an ACD device is being used on a patient and the external defibrillator/monitor arrives at a later time, it may be advantageous for the external defibrillator/monitor to easily be applied to the patient and put in communication with the ACD device without disrupting the ability for rescuers to use the ACD device in providing ACD CPR treatment. Connection interfaces and circuits described herein provide the ability for such advantages to become reality. Described in further detail below, such methods of connection may involve a mechanical connector from the external defibrillator/monitor being plugged into the ACD device, establishing data communications there between. In some cases, such a connector may have been initially plugged into another compression device, such as a chest compression sensor equipped with an accelerometer or other motion sensor, and unplugged so that a subsequent connection may be established with the ACD device. It should be understood that other methods of communicative connection may be established, without need for a mechanical connector, for example, wireless connection protocols may be used for establishing connection between the ACD device and the external defibrillator/monitor.

In an alternative scenario, a first responder may initiate resuscitative care with an external defibrillator. For example, the first responder may take an automated external defibrillator (AED) available at the scene of the cardiac arrest patient and apply it to the patient. An electrode assembly of the AED may include a chest compression pad configured for standard manual chest compressions. The AED may instruct the first responder to initiate standard manual chest compressions using the chest compression pad to obtain motion signals so that the AED is able to provide chest compression depth and rate feedback for the person applying the chest compressions. This manual chest compression pad may include one or more sensors and the external defibrillator may be connected to the sensor in order to provide the data collection, analysis, and/or display.

A second responder may arrive subsequently with an ACD device and appropriate equipment that may be used to apply the ACD device to the patient (e.g., ACD device landing pad) and continue the resuscitative care by providing ACD chest compressions in lieu of the manual chest compressions. In order to maintain the data collection, analysis, and/or display by the external defibrillator, it is desirable that a connection interface be quickly and easily transferred from the manual chest compression pad to the ACD device. Hence, a connection interface initially plugged into the manual chest compression pad may be disconnected therefrom and subsequently connected to the ACD device, so that data from the ACD device may then be accessible to the defibrillator. Such a system may allow for seamless merging of data collected from different device components into a single care record for the patient.

As a further example, the first responder may have both pieces of equipment at his/her disposal. However, this responder may follow rescue protocols that include an initiation of chest compressions prior to delivery of the defibrillation shock. As such, the ACD device may be in use prior to the application and/or use of defibrillation electrodes. The timing and arrival of various responders are examples only and are not necessary for the implementation of the systems and techniques described herein. The features of motion sensor apparatuses described herein are relevant and desirable during equipment transitions irrespective of any personnel transitions.

In any of the above described situations, it is desirable that the transitions between number and/or types of equipment in use occur efficiently and without the loss of data and/or time. Thus, the techniques and systems described herein enable the rescuer to use the ACD device without disturbing and/or rearranging defibrillation electrodes that may already be in place on the chest of the patient. Similarly, these techniques and system enable the rescuer to use the defibrillation electrodes without disturbing and/or rearranging components of the ACD device that may already be in use.

Other capabilities may be provided and not every implementation according to the disclosure must provide any, let alone all, of the capabilities discussed. Further, it may be possible for an effect noted above to be achieved by means other than that noted and a noted item/technique may not necessarily yield the noted effect.

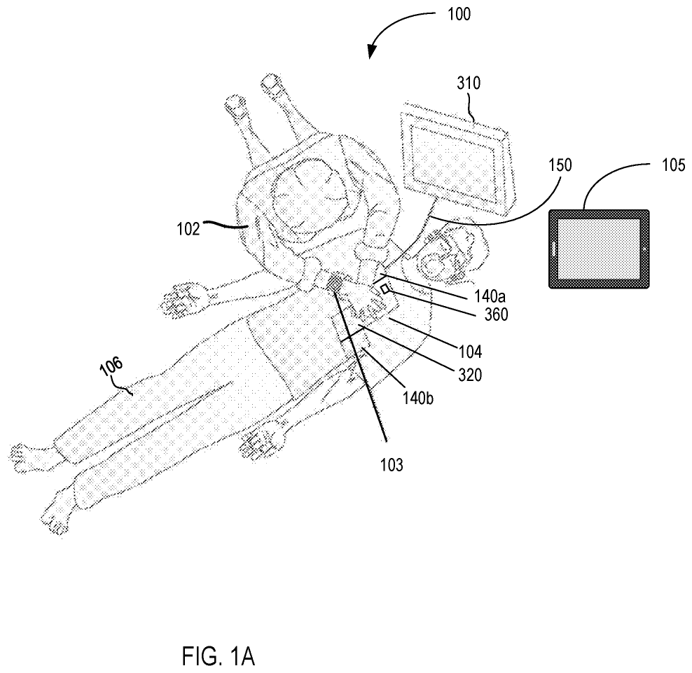

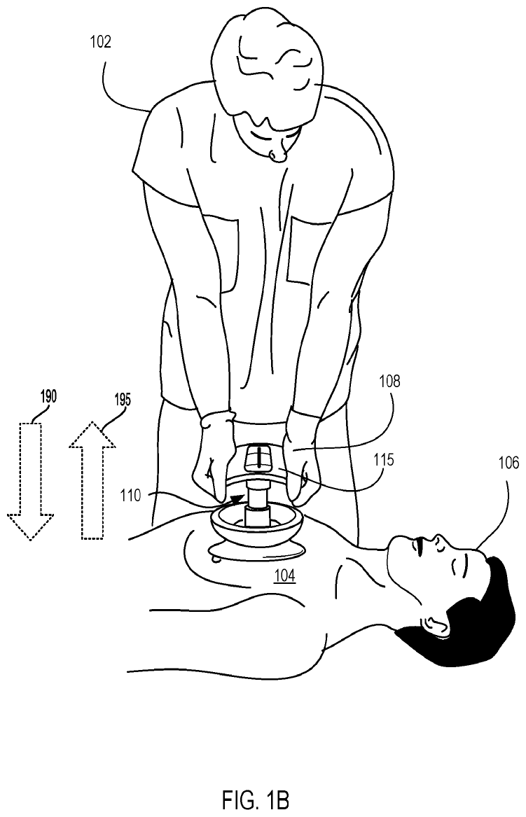

FIG. 1A is a schematic view of a rescuer 102 performing CPR chest compressions on a patient 106 using a manual compression pad 320, which is placed on the chest 104 of the patient 106. Although only one rescuer 102 is shown, multiple rescuers may participate in resuscitation activities for the patient 106.

The manual compression pad 320 includes one or more sensors 360 to generate signals indicative of CPR chest compressions of the rescuer 102, for providing CPR feedback to the rescuer. Examples of sensors that be disposed in, or on, the manual compression pad include acceleration sensors (or accelerometers), magnetic field sensors, gyroscopic sensors, proximity sensors, optical sensors, rotation and/or tilt sensors, position sensors, and gravity sensors, to list a few examples. Additionally, the one or more sensors may include a combination of two or more the above identified sensors. The signals generated from the sensor(s) may processed by a patient monitor 310 (e.g., external defibrillator) connected to the manual compression pad 320, to determine a number of motion related CPR information, such as the initiation of chest compressions, chest compression depth, chest compression rate, amongst others. Such information may then be used by the patient monitor 310, or the manual compression pad itself, to provide feedback for the rescuer to apply chest compressions according to desired depth and rate (e.g., in adherence to guidelines provided by the American Heart Association, between 2.0-2.4 inches of depth, between 100-120 compressions per minute).

In the illustrated example, the rescuer 102 utilizes a wrist-worn, mobile computing device 103, such as a smartwatch (e.g., a device that includes enhanced functionally beyond time keeping and described in more detail below). In certain embodiments, the wrist-worn, mobile computing device 103 is able to provide real-time feedback (e.g., from the patient monitor 310) to the rescuer 102 during performance of the CPR.

Control and coordination for the resuscitation event and the delivery of various therapies may be accomplished by a second device 105 or processing element that is external to the patient monitor 310, such as by use of a tablet-based computer that is controlled by the rescuer (or by a second rescuer). For instance, the second device 105 may download and process ECG data from the patient monitor 310, analyze the ECG signals, perform relevant determinations based on the analysis, and control the patient monitor 310 or other therapeutic devices (e.g., mechanical ventilators, automated chest compression device, and ultrasound transducers). Additionally, the second device 105 may mirror the display of the patient monitor to enable a more senior rescuer to monitor the activity of the junior rescuer.

In FIG. 1a, an electrode 140a is positioned high on the right side of the patient's torso and a separate electrode 140b is positioned low on the left side of the patient's torso so that the electrodes 140a, 140b form an electrical vector that extends through the patient's heart. The manual compression pad 320 is placed at a sternal position of the chest 104 of the patient 106. The electrodes may include electrocardiogram (ECG) sensors to measure electrical activity of the heart.

The patient monitor 310 in this example is connected to both electrodes 104a, 140b via a single cable 150. In alternative embodiments (as detailed below), each electrode connects to the patient monitor via a separate cable. The patient monitor 310 may take a generally common form, and may be a professional style defibrillator, such as the X Series, R Series, M Series, or E Series from ZOLL Medical Corporation of Chelmsford, or similar variants thereof. Massachusetts. Alternatively, the patient monitor 310 may be an automated external defibrillator (AED), including the AED Plus, or AED Pro, from ZOLL Medical Corporation, or similar variants thereof.

Relevant information from the patient monitor 310 may be transmitted to or from the wrist-worn mobile computing device 103. For example, information can be visually presented on a display of the wrist-worn mobile computing device 103. Additionally, haptic feedback (e.g., vibrations) or audible feedback (e.g., tones, buzzers, or alarms) can also provide feedback to the rescuer 102. Different types of audible or haptic feedback can be utilized to provide indication of specific events. For example, a constant metronomic tone or vibration may be utilized to provide an indication for a rate of chest compressions. Likewise, an alarm or continuous vibration may indicate that the some vital is below a predefined threshold (e.g., heart rate too low, blood pressure too low, lack of a heartbeat (asystole) or erratic heartbeat, oxygen saturation below a threshold, etc.). Additionally, still another different visual, audible, or haptic feedback may provide an indication that a ventilation is required or that a therapeutic agent is required, to list additional examples. The different types of alarms may be pre-programed based on current industry or regulatory guidelines. Additionally, the alarms may be user-modifiable.

In the illustrated example, the wrist-worn mobile computing device 103 is a wrist-worn device, commonly referred to as a smartwatch (e.g., a computerized wristwatch with functionality enhanced beyond timekeeping). Such a smartwatch can effectively be a wearable computer. The smartwatch can include a data processor, memory, input (buttons and/or touchscreen), and outputs (display, speakers, vibrations). The smartwatch may additionally collect information from internal sensors to monitor the rescuer or to measure activities performed by the rescuer. The smartwatch may control or retrieve data from the patient monitor 310 or second device 105, other instruments or computers located at the rescuer event. The smartwatch may support wireless technologies, like 3G/4G/5G network protocols, Bluetooth, near field communication (NFC), and/or Wi-Fi, to communicate with the patient monitor 310, second device 105 or an emergency response center (e.g., dispatch or hospital).

In other examples, the smartwatch may just serve as a front end for a remote system and be configured to display information generated by the patient monitor 310. The display can be made of Indium gallium zinc oxide (IGZO), a semiconducting material. IGZO thin-film transistors (TFT) can be used in the TFT backplane of flat-panel displays (FPDs). Because the IGZO display is flexible, a greater amount of information can be displayed on the wrist-worn devices due to the increased surface area of the display.

FIG. 1B is a schematic diagram of an Active Compression/Decompression (ACD) device 110 in use on a patient 106.

In the illustrated example, the ACD device 110 is affixed directly to the chest 104 of the patient 106. The rescuer 102 holds a handle 115 of the ACD device 110 with his/her hands 108 and manually operates the ACD device 110 to actively compress and actively decompress the chest 104 of the patient 106. The rescuer 102 applies a compression force 190 (e.g., a downward force) to compress the chest 104 of the patient 106. Then, when the rescuer 102 pulls the handle 115 of the ACD device 110, a decompression force 195 (e.g., an upward force) actively decompresses the chest 104.

Active compression and active decompression further enhances circulation throughout the body. For instance, active compression results in the application of positive intrathoracic pressure, leading to the ejection of blood out of the ventricles and away from the heart (and thus delivery of oxygen to other organs). Active decompression, on the other hand, results in negative intrathoracic pressure, which enhances venous return of blood back to the heart. In the absence of active decompression, the chest passively returns to a neutral position during the release phase (i.e., the decompression phase) of the chest compression cycle. A neutral position is defined as a position of the sternum when no force, either upward or downward, is applied to the chest. The exertion of the upward force (i.e., the active decompression) may increase the release velocity associated with the decompression as compared to the release velocity without active decompression. Such an increase in the release velocity may increase the negative intrathoracic pressure and thereby enhance venous flow into the heart and lungs from the peripheral venous vasculature of the patient. In other words, the active decompression may enhance venous return of blood to the heart to refill the cardiac chambers. The active decompression may also enhance ventilation in the patient's lungs.

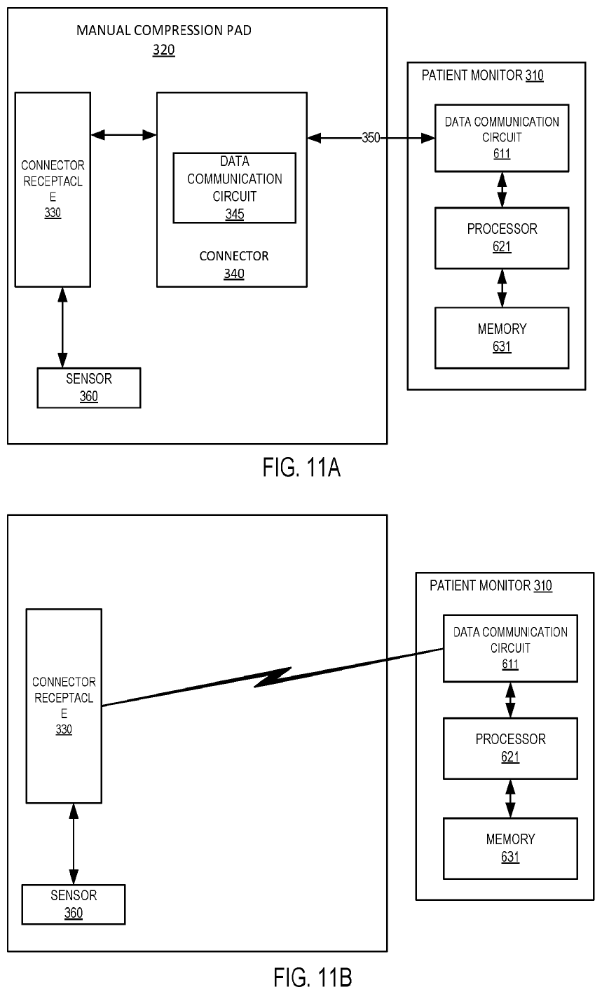

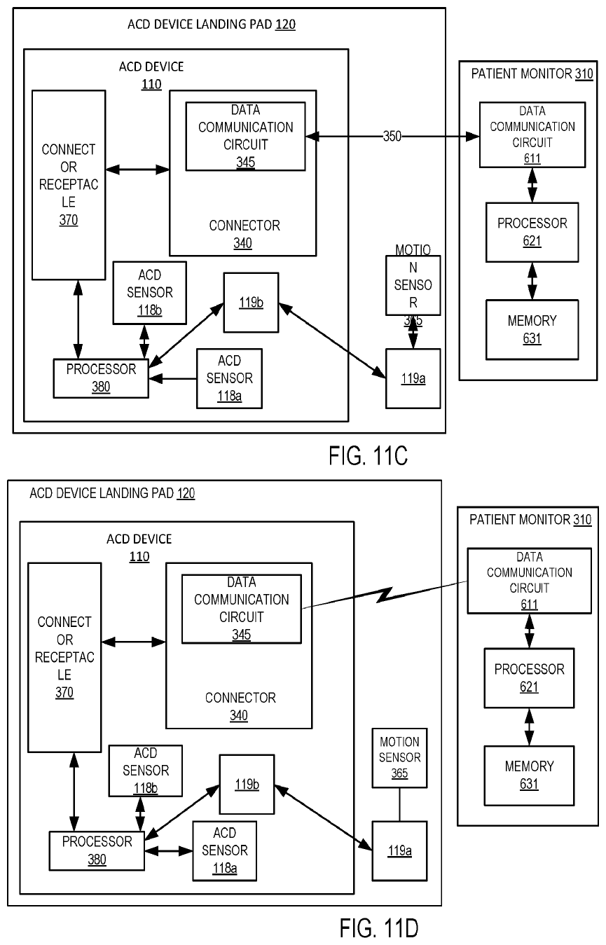

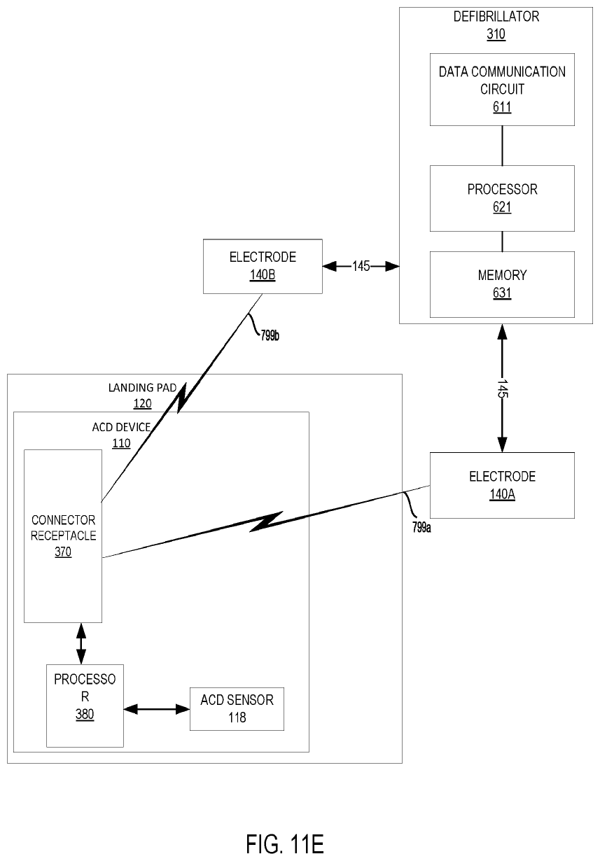

Additionally, during ACD chest compressions the compression phase and decompression phase will both have a portion of motion during which the sternum is compressed past a neutral position and pulled upward beyond the neutral position. In order to determine chest compression depths during compressions, a waveform analysis is performed (e.g., by the processor 621 of patient monitor 310 shown in FIGS. 11A-11E) to identify the neutral point. In an implementation, the algorithm may set a pre-compression neutral point as the initial position of the chest prior to an initiation of chest compressions.

Additionally, due to chest remodeling, which typically occurs during chest compressions, the neutral point may change over the course of applied chest compressions. Chest remodeling generally refers to changes in the anterior/posterior diameter of the patient's chest based on a combination of an applied force during the chest compressions and a compliance of the patient's chest. Chest compliance is the mathematical description of the tendency of the chest to change shape as a result of the applied force. Thus, compression depth feedback based on the pre-compression neutral point is likely to be inaccurate.

In order to provide accurate compression depth feedback, the processor of the patient monitor 621 may be configured to dynamically determine the neutral point to account for changes in the compression neutral point over the course of chest compressions. To this end, the waveform analysis algorithm may need additional information such as compression force information (e.g., as provided by the one or more force sensors in the ACD device), motion information for the elevated and non-elevated phases, and chest compliance information. The chest compliance information may be a mathematical relationship between displacement, force, and chest compliance.

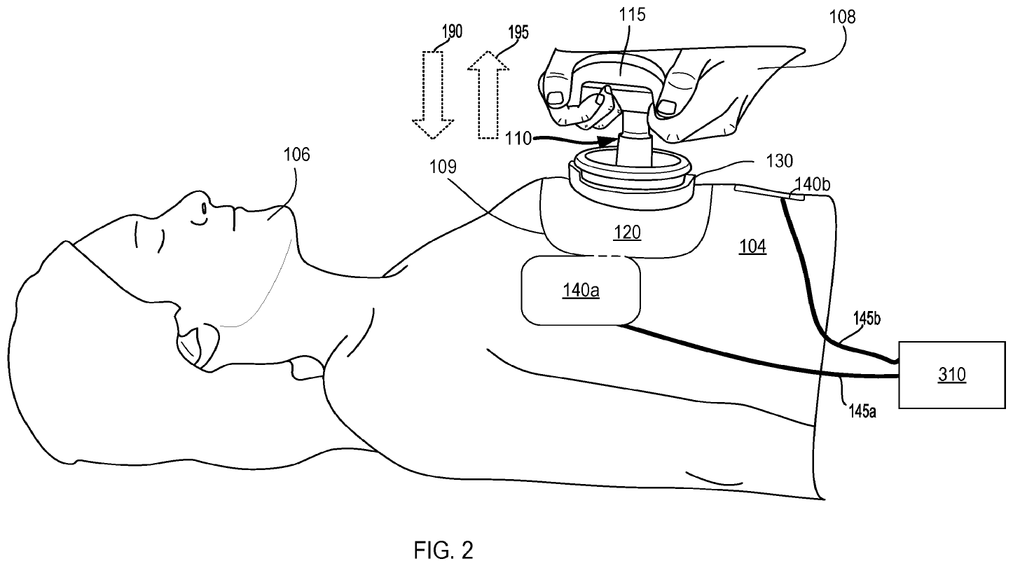

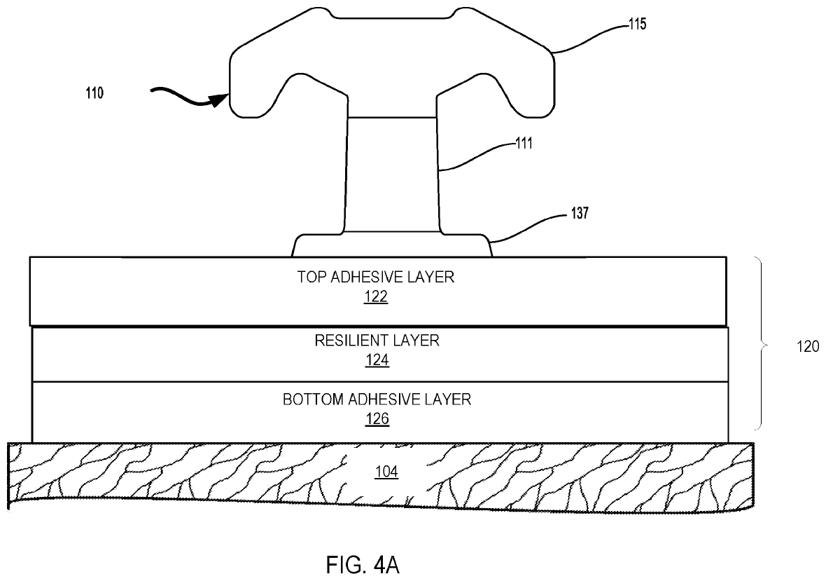

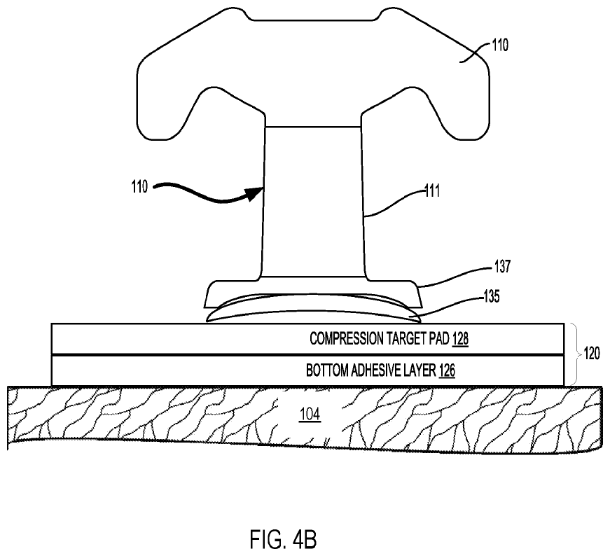

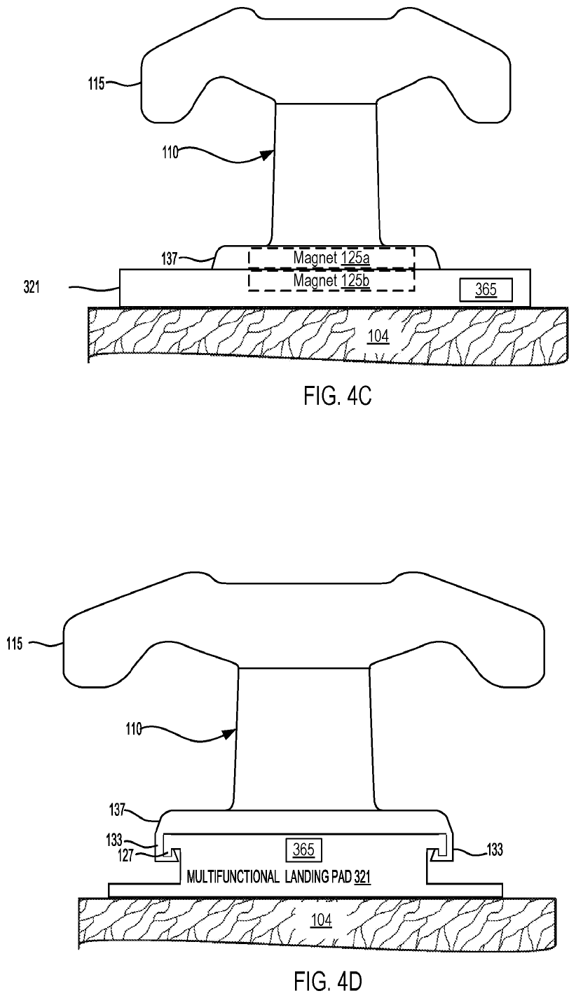

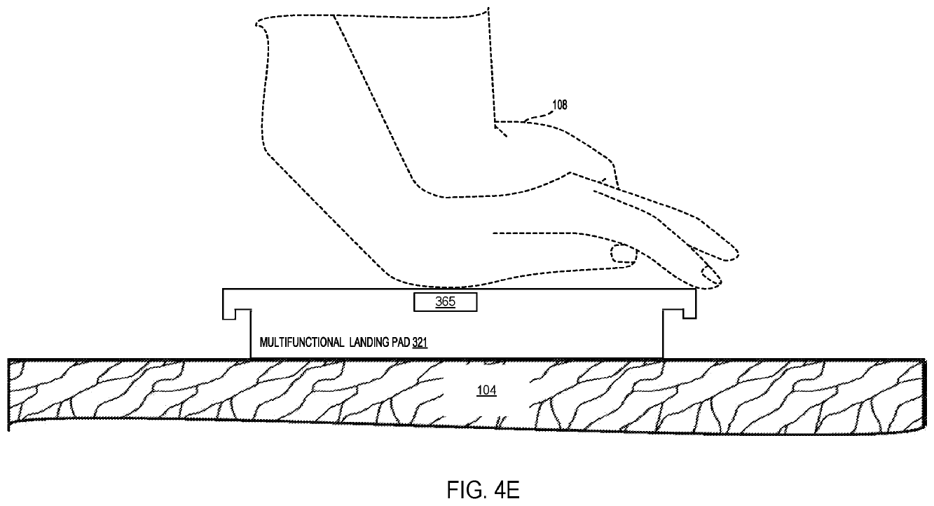

Referring to FIG. 2, the ACD device 110 may be component of a system that further includes an ACD device landing pad 120 (or landing pad 120), the patient monitor 310, and an optional ACD device mount 130. It should be noted that the ACD device mount 130 described in FIG. 2 is non-limiting example. Additional method of affixing the ACD device to the patient as well as additional examples of device mounts are described in FIGS. 4A-4D. The ACD device landing pad 120 may be attached to the chest 104 of the patient 106, such as via an adhesive that is able to exert an upward force on the chest of the patient as the ACD device 110 is pulled upward. In one example, the ACD device 110 is affixed directly to the landing pad 120. In another example, the ACD device engages with the ACD device mount 130 that engages a base portion of the ACD device and “locks” the ACD device into the ACD device mount 130 so that force exerted on the ACD device 110 is transferred through the mount 130 and to the landing pad 120. In some embodiments, as discussed further herein, the ACD device mount 130 may be provided as a multifunctional compression pad, which allows for both standard manual chest compressions and engagement of an ACD device for ACD treatment to be applied. For instance, if the ACD device is not yet engaged with the ACD mount and landing pad assembly, a rescuer may provide chest compressions on the ACD mount 130, where the ACD mount 130 incorporates a motion sensor (e.g., accelerometer) which provides motion signals for the patient monitor 310 to provide chest compression feedback in accordance with subject matter described herein. Once the ACD device is ready for use, the ACD device 110 may be affixed to the ACD mount 130, so as to allow for ACD treatment to then be applied.

FIG. 2 further illustrates the ACD device 110 being integrated with electrodes 140a and 140b of the patient monitor 310. The electrodes 140a and 140b may be defibrillation electrodes electrically coupled to an external defibrillator by high voltage cables 145a and 145b. Further, the electrodes 140a and 140b may be releasably attached to the chest 104 of the patient 106. The electrode pads 140a, 140b, which may further include electrocardiogram (ECG) sensors to measure electrical activity of the heart of the patient.

Proper placement of the electrode pads 140a, 140b on the patient 101 is important to ensure effectiveness of the therapy. In adults, one electrode pad is typically placed on the patient's right chest above their right nipple and the second electrode pad is typically placed on the left lateral side of the patient opposite placement of the first electrode pad. In pediatric patients, who are comparatively lighter in weight than adults, one electrode pad is typically placed on the front right chest wall and the second electrode pad is typically placed on the back of the thorax. As discussed previously, the electrode pads are positioned in a manner that forms an electrical vector between the electrode pads through the heart of the patient, for efficacious electrotherapy.

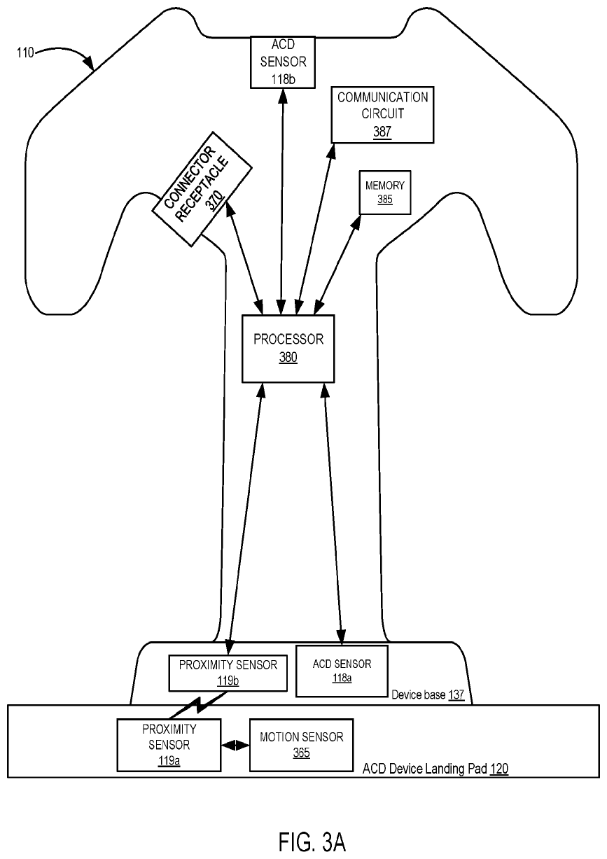

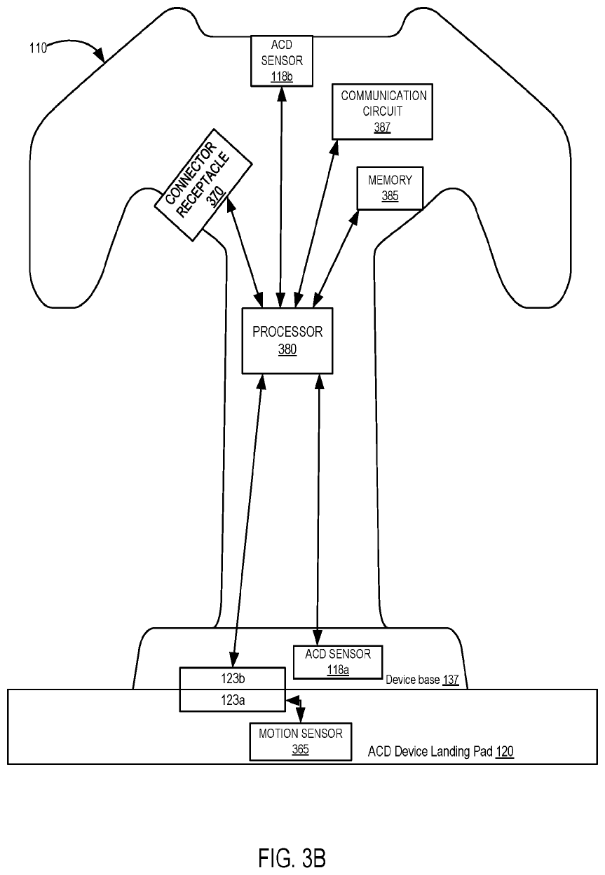

FIG. 3A is a block diagram illustrating components of an embodiment of an active compression-decompression (ACD) device 110 that is able to communicate wirelessly with the ACD device landing pad 120.

ACD sensors 118a, 118b of the ACD device 110 are configured to sense parameters related to the force applied by a rescuer 102 using the ACD device 110. While the sensors are identified as ACD sensors, they could be any suitable types of sensors such as pressure sensors, force sensors, acceleration sensors (or accelerometers), and/or magnetic field sensors, to list a few examples. The sensors 118a, 118b are configured to generate signals indicative of motion and/or force applied due to chest compressions. In an example, ACD sensors 118a, 118b are force sensors for measuring force applied to the handle and force applied to the base. In another embodiment, ACD sensor 118a is a motion sensor for measuring displacement (or acceleration or velocity) at the base, and ACD sensor 118b is also a motion sensor for measuring displacement (or acceleration or velocity) at the handle. In another embodiment, ACD sensor 118a is a force sensor for measuring force at the base, and ACD sensor 118b is a motion sensor for measuring displacement (or acceleration or velocity) at the handle. In another embodiment, ACD sensor 118a is a motion sensor for measuring displacement (or acceleration or velocity) at the base, and ACD sensor 118b is a force sensor for measuring force at the handle.

In one example, the generated signal may be in response to the rescuer 102 pushing on and releasing the chest 104 of the patient 106 with the ACD device. However, the sensors 118a, 118b may also generate “artifacts” induced by movement of a support structure such as a gurney and/or by motion of a transport vehicle such as an ambulance. Likewise, the sensors 118a, 118b may also generate motion signals when a rescuer 102 is positioning the ACD device 110 on the chest 104, for example.

A processor 380 of the ACD may include algorithms configured to combine the signals from the motion sensor(s) 365 of the landing pad 120 as well as the signals obtained by the ACD sensors 118a, 118b that correspond to repetitive chest compressions and decompressions. The ACD device further includes memory 385 for storing data as well as executable instructions used by the processor 380. The ACD device further includes a connection receptacle 370 for receiving a connector 340 (described in further detail below) to enable the ACD device 110 to interface with the patient monitor 310, or other external devices (e.g., device 105). Additionally, the processor may include and/or be connected to a communication circuit 387, which enables the ACD device to communicate via by cabled connection (e.g. USB, RS232) or wireless (NFC, low power Bluetooth, 802.11) to external devices. As discussed, for some embodiments, the connector 340 may be unplugged from a manual compression pad and connected with the connector receptacle 370 so as to establish a communications link from the ACD device with another device.

As noted herein and discussed in further detail, the ACD device 110 may be placed in suitable engagement with the ACD device landing pad 120, so that upward or downward force applied to the handle of the ACD device 110 is transferred to the ACD device landing pad 120 and ultimately to the patient's chest. Hence, the device base 137 may be structured for such engagement with the landing pad 120, for example, via locking components, adhesive, magnetic features, etc.

Additionally, one or more sensors (e.g., motions sensors, accelerometers, force/pressure sensors) 365 are disposed on the ACD device landing pad 120. As one example, the one or more sensors may be a motion sensor 365 that may be operably connected to, for example, a proximity sensor 119a (e.g., near field communication (NFC) tag and antenna, RFID), which communicates to a corresponding proximity sensor 119b (e.g., appropriately configured NFC tag, RFID) located in the base portion 137 of the ACD device 110. Taking a NFC tag as an example of a proximity sensor that may be used in embodiments described herein, NFC is a short range (i.e., 10 centimeters or 4 inches) communication protocol that allows communication of data between compatible device (e.g., devices with appropriately configured NFC tags). This wireless communication enables any signals measured and generated by the sensor 365 in the landing pad to be communicated to the processor 380 the ACD device.

As a result, as the ACD device 110 and the landing pad 120 are placed in appropriate mechanical engagement with one another, the proximity sensors 119a, 119b may also initiate mutual communication so that data is able to be transferred between the landing pad 120 and the ACD device 110. In various embodiments, wireless communication between an ACD device, or landing pad for an ACD device, and a patient monitor or defibrillator may be enabled, capable of being enabled, or established, such as automatically and without requiring any user action specifically to establish, or related exclusively to establishing, the wireless communication, when the devices are sufficiently proximate to each other, or when one of the devices comes or is brought sufficiently proximate to the other device, for example. As an example, motion signals obtained from the motion sensor 365 may be transmitted to the processor 380 for further processing and/or pass through. Further signal transmission may then occur between the processor 380 and the connector receptacle 370, which is then transmitted externally, for example, to patient monitor 310. In some embodiments, the processor 380 processes data obtained from the ACD sensor(s) 118a, 118b, so that relevant ACD feedback, other types of prompting or feedback, or storage for reporting purposes, may be provided for the user(s) of the system. Alternatively, or in addition, the patient monitor may process such data obtained from the ACD sensor(s) 118a, 118b, for similar reasons, e.g., provide ACD feedback or data reporting/recording from the emergency event.

In some embodiments, as noted above, before the ACD device 110 is ready for use, it may be preferable for a rescuer to apply standard manual chest compressions to the patient. In this case, the landing pad 120 may be positioned on the patient with the motion sensor 365 located at the sternum so that a rescuer may provide standard manual chest compressions with the benefit of signals generated from the motion sensor 365. Accordingly, while not expressly shown in this figure, the landing pad 120 may be in communication with the patient monitor 310 so that signals from the motion sensor 365 may be processed in a manner that results in chest compression feedback being provided to the rescuer. Once the ACD device 110 is placed, signals from the ACD sensor(s) 118a, 118b and/or the motion sensor 365 may then be used to generate feedback suitable for ACD treatment, in accordance with embodiments discussed herein.

FIG. 3B is similar to FIG. 3A, except in this embodiment, there is a mechanical interface connection 123a on the landing pad (or within the ACD device mount 130) and a corresponding interface connection 123b in the base portion 137 of the ACD device 110 to enable a direct electrical connection there between. In this case, rather than the connection being established through proximity (e.g., via NFC tags or RFID), communications are able to occur through the conductive contacts of interface connections 123a, 123b.

FIGS. 4A-4D illustrate various configurations for affixing the ACD device 110 to the ACD device landing pad 120 which may otherwise be used as a multifunctional landing pad 321. While none of FIGS. 4A-4D illustrate the ACD device mount 130 as shown in FIG. 2, it is understood that any of the illustrated examples could be modified to include the ACD device mount 130 affixed to the landing pad 120, which would then engage a base portion 137 of the ACD device 110, for appropriate mechanical coupling therewith. That is to say, there are several possible configurations for attaching the ACD device to the landing pad and the patient, and these figures should be viewed as non-limiting that may modified or combined.

Referring to FIG. 4A, according to some embodiments, the handle 115 of the ACD device 110 is coupled to a shaft 111, which is further coupled to a base portion 137. Alternatively, the shaft and base portion may be a single component collectively identified as the shaft. The base portion then is connected to the ACD device landing pad 120, which is enlarged for clarity. The shaft 111 may or may not include a sensor disposed within the shaft. The landing pad 120 includes a top adhesive layer 122, a resilient layer 124, and a bottom adhesive layer 126. The top adhesive layer 122 includes a bonding agent on the surface of the landing pad that causes the landing pad to adhere to the base portion 137 of the ACD device 110 during chest compressions. The top adhesive layer provides enough adhesion to keep the ACD device from separating from the landing pad 120 during active decompressions (i.e., pulling upward). Similarly, the bottom adhesive layer 126 adheres to the chest 104 of the patient 106. Likewise, the bottom adhesive layer also provides enough adhesion to prevent the ACD device, which is adhered to the landing pad 120, from separating the landing pad 120 from the chest 104 of the patient 106. In some examples, the resilient layer 124 may be a closed-cell polyethylene foam such as a Volara™ foam provided by Sekisui Voltek, with a thickness of 0.125 inches. The resilient layer may include other materials as well.

The landing pad 120 shown in FIG. 4B is similar to the landing pad described with respect to FIG. 4A, in this embodiment, however, the landing pad 120 does not include a top adhesive layer. Rather, a substantially flat, non-porous surface (e.g., compression target pad 128) is provided for attachment of a suction cup 135 of the ACD device. In the illustrated embodiment, the base portion 137 of the ACD device 110 is concave and houses a suction cup 135. The suction cup 135 includes a concave area which traps air inside of it when affixed to the (generally) flat non-porous surface of the ACD device. Once air is trapped, a vacuum is created which causes the ACD device 110 to remain attached to the landing pad 120 during chest compressions unless (or until) the seal between the suction cup and landing pad is broken. In a typical implementation, the suction cup is made from silicon or soft rubber, for example. While not illustrated, the suction cup may include a small pump to pump out of the air of the suction cup.

FIG. 4C illustrates an alternative example of the how ACD device 110 may connect to a multifunctional chest compression pad 321. The multifunctional chest compression pad 321 is a chest compression pad that can be used as a manual chest compression pad (for standard manual chest compressions with the hands), while also allowing for the secure attachment of the ACD device 110, for ACD therapy to be provided to the patient in place of standard manual chest compressions. Hence, the multifunctional landing pad 321 may be adhesively attached to the chest of the patient.

FIG. 4E illustrates an example in which the multifunctional landing pad 321 is used without an ACD device (i.e., as a manual compression pad). And as illustrated, the top surface of the multifunctional chest compression pad 321 is generally flat and provides a surface on which standard manual chest compressions (with the hands 108) may be performed. Similar to previous embodiments, one or more sensors 365 are disposed in, or on, the landing pad to generate signals indicative of chest wall motion. These signals for tracking chest wall motion may be transmitted to a chest compression feedback device, such as a patient monitor and/or defibrillator, secondary display screen (e.g., tablet, mobile computing device), so that appropriate chest compression feedback for rate and depth may be provided for the rescuer.

When a compatible ACD device arrives, the ACD device may be suitably attached to the multifunctional compression pad so that ACD treatment is able to be provided. That is, when ready for use, the rescuer may push the ACD device into the patient upon compression downstroke of ACD treatment, followed by pulling upward of the ACD device from the patient during the decompression upstroke. When the decompression phase passes the neutral point on the upstroke, the rescuer pulls upward on the ACD device, which in turns pulls upward on the patient's chest via adherence of the multifunctional compression pad thereto.

In this example, for attachment of the ACD device with the multifunctional compression pad, one or more magnets 125a are located both in a base portion 137 of the ACD device 110 and one or more corresponding magnet 125b are located within the multifunctional chest compression pad 321. In general, the magnetic fields (and attractive forces) created by the magnets must be strong enough to keep the ACD device from separating from the multifunctional chest compression pad 321 during ACD CPR therapy. For example, the magnets could be rare-earth magnets such as neodymium magnets, which are one of the strongest commercial available permanent magnets.

FIG. 4D illustrates yet another example of how to attach the ACD device 110 to the multifunctional chest compression pad 321. The multifunctional landing pad 321 may be adhesively attached to the chest of the patient. In the illustrated example, the multifunctional chest compression pad 321 includes flat top surface on which manual chest compressions may be performed. Additionally, the landing pad may include mechanical features, such as a lip (or ridge) 127 that extends about the perimeter (or circumference) of the landing pad 120. The base portion 137 of the ACD device 110 includes corresponding “press fit” (or “snap fit”) latches 133 configured to engage the lip 127 upon the application of force and secure the ACD device 110 to the multifunctional chest compression pad 321.

In operation, the rescuer 106 orients the ACD device 110 such that the base 137 is in alignment with corresponding coupling features (e.g., mechanical, magnetic) of the multifunctional landing pad 321. In the embodiment of FIG. 4D, the rescuer may then push downward on the ACD device 110 causing the semi-rigid press fit latches to spread outward (i.e., away from the landing pad 120). Once the latches 133 clear the ridge 127 the latches 133 will return to their original position (i.e., move toward the landing pad 120) resulting in the press fit latches 133 locking with landing pad 120 (i.e., docking). Upon successful docking, both the ACD device 110 and landing pad will move in conjunction with one another in response to any upward or downward motion of the ACD. Thus, any motion of the ACD device 110 will be also imparted onto the landing pad 120 and chest of the patient.

Alternatively, the latches 133 may pass through a plurality of openings within the multifunctional chest compression pad 321. In this embodiment, the latches would be compressed “inward” (i.e., toward the multifunctional chest compression pad) while passing through the openings and then expand “outward” once the latches passed through the opening to secure the ACD device 110 to multifunctional chest compression pad 321. It should be appreciated that any other suitable mechanical coupling may be employed for securing the ACD device 110 to the multifunctional landing pad 321, for ACD therapy to be provided to the patient.

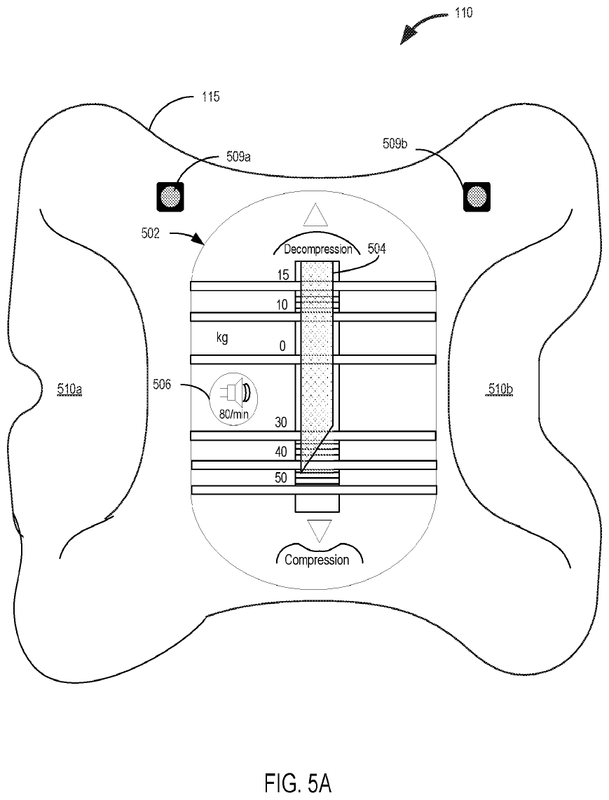

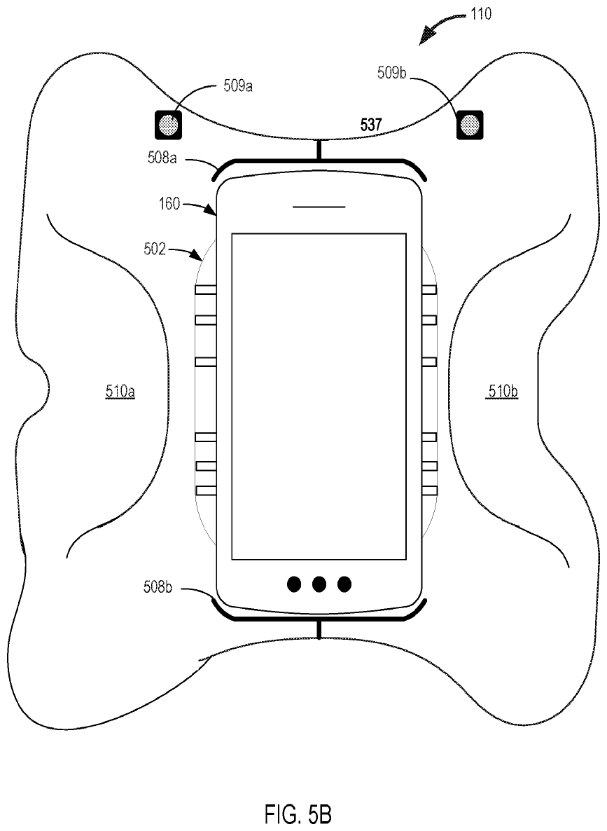

FIG. 5A illustrates one example of a display 502 mounted on or otherwise provided with the handle 115 of the ACD device 110. As an example, the display 502 may be integrated with the handle upon manufacture. In a typical implementation, the rescuer 102 places their hands 108 on the side portions 510a, 510b of the handle 115. A force or displacement gauge 504 displays the amount of compressive (downward) and decompressive (upward) forces or displacement applied to the chest of patient 106 during chest compressions and decompressions in real-time. In various embodiments, the force and displacement sensor(s) provided with the ACD device may measure the force and displacement associated with administration of ACD treatment to the patient. Signals associated with such treatment may be received and processed by the processor of the ACD device, so that the display 502 provides the appropriate feedback. Force and displacement signals may also be transmitted to a corresponding patient monitor and/or compression feedback device to which the ACD device is in communication, for processing and presentation of feedback for ACD treatment on the patient monitor or other compression feedback device. In various embodiments, feedback may be employed similar to that described in US Patent Publication No. 2018/0092803, filed on Sep. 29, 2017 and entitled “Active Compression Decompression Cardiopulmonary Resuscitation Chest Compression Feedback,” the disclosure of which is incorporated by reference herein in its entirety. Additionally, the handle 115 further includes a speaker 506 that provides audible feedback. In one example, metronomic tones are provided at a rate, for example, of 80 compressions per minute (or any other suitable compression rate) via the speaker 506 to provide guidance of the proper compression rate.

One or more lights 509a, 509b may be disposed in the handle 115 and illuminate at an appropriate frequency (e.g., approximately 10 times per minute), depending on the protocol (e.g., 30:2 compressions to ventilations, 15:2 compressions to ventilations, continuous compressions, amongst others) to provide an indication of when to ventilate the patient. Alternatively, the lights may be used as visual metronomes to provide an indication of when to compress or decompress. Ventilations may be provided mouth-to-mouth, with a bag-valve-mask ventilation device, or with a mechanical ventilator. Currently, the chest compression rate for standard manual chest compressions (with the hands) is typically approximately 100-120 compressions per minute. However, a typical chest compression rate with the ACD device is often 80 compressions per minute, for example, due to the increased efficiency of the ACD device, which is able move greater volumes of blood. The slightly slower compression rate with the ACD device, as compared to manual chest compressions, may provide more time for the heart accommodate the increased filling and ejection of blood during compression with ACD device.

FIG. 5B illustrates an example of the ACD in which the ACD device is used in conjunction with a portable computing device 160, such as a smartphone. In this embodiment, the display 502 is covered by the portable computing device 160, which provides additional functionality such as the ability to communicate with rescue services, emergency dispatch, a hospital or possibly even rescuers enroute. The portable computing device 160 may provide visual, audible, or haptic feedback, or coaching, or the ability to input information (e.g., via keyboard or with voice-to-text) to the ACD device. Additionally, the portable computing device may provide step by step instructions for CPR or application of defibrillation electrodes, to list a few examples.

In the illustrated example, the portable computing device 160 is mounted to brackets 508a, 508b disposed on the handle 115 of the ACD device 110. The brackets 508a, 508b may provide electrical contacts for communication of the portable computing device and the ACD device. Additionally, in some embodiments, a camera of the portable computing device 160 may be used to capture audio and/or video of the rescue scene and transmitted to the rescue services.

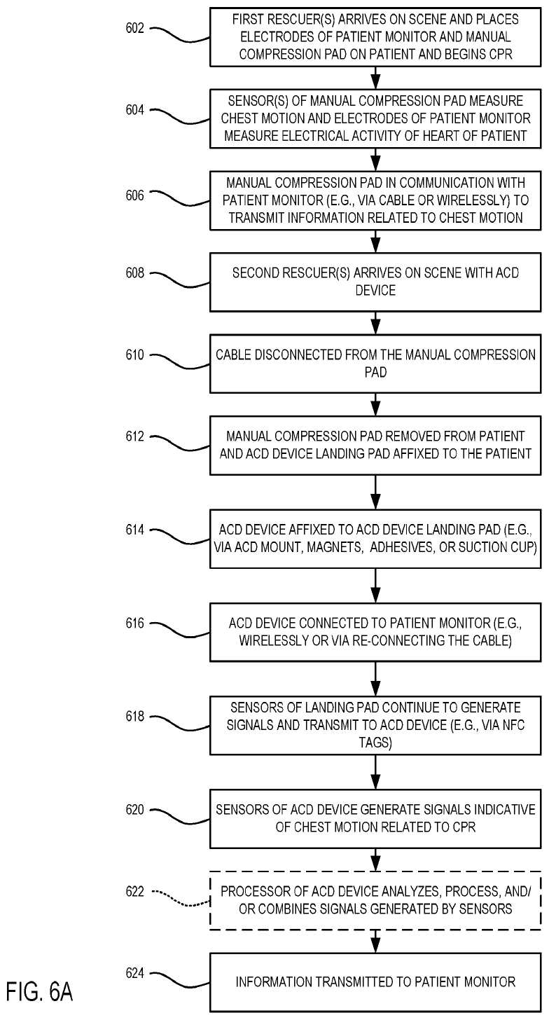

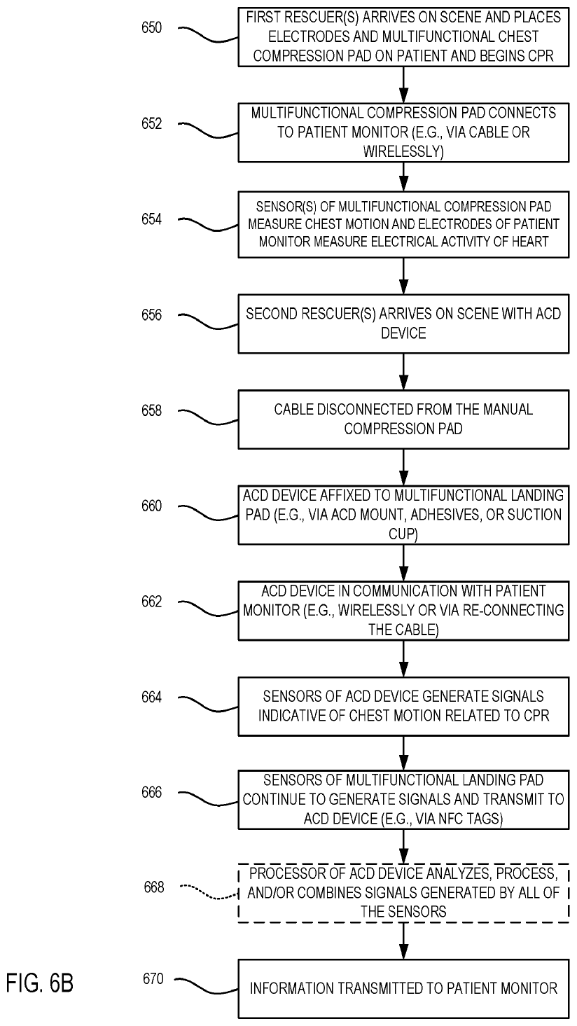

FIG. 6A is a flow chart of an embodiment illustrating the steps performed by rescuers during a rescue event in which rescuers swap a manual chest compression pad for the ACD device landing pad, so that the ACD device may be used and integrated with the patient monitor and, hence, the overall emergency event. In this case, a defibrillator is available, however an ACD device is not yet available. Accordingly, the electrodes of the defibrillator and the manual compression pad associated therewith will first be placed on the patient, only to be replaced with appropriate equipment for ACD (e.g., ACD device with landing pad, integrated therewith or separately available).

Referring to FIG. 6A, in step 602, a first rescuer (or rescuers) arrives on scene and places the electrodes 140a, 140b of the patient monitor 310 (e.g., defibrillator) and a manual compression pad 320 on the patient 106 and the rescuers begins performing CPR. In various embodiments, the electrodes and manual compression pad are provided together, for example, attached to one another out of the package. Next, in step 604, the motion sensor (e.g., accelerometer) of the manual compression pad 320 measures chest wall motion of the patient during the performance of standard manual chest compressions with the hands. Likewise, the electrodes 140a, 140b detect and measure the electrical activity of the heart (ECG data) of the patient 106. In step 606, the manual compression pad 320 is in communication with the patient monitor 310 via a cable 150 in order to transmit the chest motion information and the ECG data to the patient monitor 310, so that appropriate feedback may be provided by the patient monitor 310.

The second rescuer (or rescuers) arrive on scene with the ACD device 110 and the landing pad 120 in step 608. In step 610, the cable 150 is disconnected from the manual compression pad 320, so that suitable data communications may be imminently established between the patient monitor and the ACD device. Then, the manual compression pad is removed from the chest 104 of the patient 106 and the ACD device landing pad 120 is affixed to the patient in step 612. In step 614, the ACD device 110 is affixed to ACD device landing pad 120 (e.g., using connections methods described in FIGS. 4A-4D). However, it can be appreciated that in some embodiments, the ACD device 110 and landing pad 120 are already assembled or otherwise integrated with one another. That is, if the ACD device 110 and landing pad 120 are effectively provided as a single apparatus, it may not be necessary to perform the first step of adhering the landing pad to the patient and then attaching the ACD device to the landing pad; rather the ACD device may be adhered to the patient in a single step. Then, the ACD device 110 is connected to patient monitor via re-connecting the cable 150, allowing for data communications between the patient monitor and the ACD device (e.g., transmission of force data, displacement data, feedback information, etc.). Alternatively, the connection between the ACD device and patient monitor could also be wireless, for example, via Bluetooth protocols, 802.11 protocols, cellular communication protocols (e.g., 3G, 4G, or 5G protocols). That is, for certain embodiments, the cable 150 may not be necessary. For example, when the ACD device is appropriately situated on the patient, a wireless communication connection may be established between the ACD device and the patient monitor, for transmission of data there between.

In the next step, 618, the sensor, or sensors, 365 of ACD device landing pad 120 may continue to generate signals indicative to chest motion during the performance of CPR and transmit those signals (e.g., via NFC tags disposed within the landing pad and ACD device or via the interface connections as detailed in FIGS. 3A and 3B. Similarly, in step 620, the one or more sensors 118a, 118b of ACD device 110 also generate signals indicative of chest motion during the performance of CPR via the ACD device 110. In an optional step 622, the processor 380 of the ACD device 110 may analyze, process, filter, and/or combine the signals generated by all of the sensors. Lastly, in step 624, all of the information is transmitted from the ACD device 110 to the patient monitor 310 via the cable connection 150.

FIG. 6B is a flow chart illustrating the steps performed by rescuers during a rescue event in which the rescuer (or rescuers) utilize a multifunctional chest compression pad. Similar to the previous use scenario, the defibrillator is available prior to arrival of the ACD device, however, rather than having a standard manual chest compression pad available therewith, the electrodes are integrated with a multifunctional compression pad. In this case, a rescuer may have the benefit of compression feedback while performing standard manual chest compressions, yet not have to remove the compression pad from the patient's chest. Rather, the ACD device may be attached directly to the multifunctional compression pad, on which the rescuer had been performing standard manual chest compressions.

In the first step 650, the first rescuer (or rescuers) arrive on scene and places the electrodes 140a, 140b and multifunctional chest compression pad 321 on the chest 104 of the patient 106 and begin performing standard manual chest compressions using the multifunctional chest compression pad 321, which has a motion sensor (e.g., accelerometer) incorporated therein. In step 652, if not already connected, the rescuer 102 ensures that the multifunctional chest compression pad 321 is connected to the patient monitor 310 (e.g., via cable 150 or via wireless protocols). The sensors 360 of the multifunctional chest compression pad 321 generate signals indicative of chest wall motion to measure compression depth and rate during CPR. Additionally, the electrodes 140a, 140b of the patient monitor 310 measure electrical activity of heart (ECG data) of the patient 106 in step 654.

In step 656, the second rescuer (or rescuers) arrives on scene with ACD device 110. The cable 150 is disconnected from the multifunctional chest compression pad 321 in step 658, the ACD device 110 is affixed to multifunctional chest compression pad 321 in step 660, and the ACD device 110 is then connected to the patient monitor 310 via connecting the cable 150. A benefit of this embodiment is that there is no need to swap the manual compression pad for the ACD compression pad; that is, the ACD compression pad and the manual compression pad are one in the same and so there is no need for any compression pad to be removed from the patient's body during the course of CPR. This makes the transition from manual chest compressions to chest compressions with the ACD device more efficient and minimizes the length of any stoppages in chest compressions, which can be detrimental for the health of the patient.

In the next step 664, sensors 118a, 118b of the ACD device 110 generate signals indicative of chest motion related to CPR. And in step 666, the sensors 365 of the multifunctional chest compression pad 321 may continue to generate signals indicative of chest motion and transmit those signals to the ACD device (e.g., via NFC tags described in FIG. 3a). In an optional step 668, the processor 380 of the ACD device 110 may analyze, process, filter, and/or combine the signals generated by the sensors (e.g., 118a, 188b, 365). Lastly, in step 670, the information from all of the sensors is transmitted to the patient monitor 310.

While both of these embodiments are described as having 2 sets of rescuers arriving on scene, it is understood that similar scenarios would include situations in which rescuers transport the patient to a location with a second set of rescuers (e.g., from the location of an accident to an ambulance, from an ambulance to hospital, or from one hospital to another hospital). Likewise, another example would be a scenario in which the second rescuer is part of a team with the first rescuer and the first rescuer begins chest compressions prior to the application of the ACD device. In yet another example, the situation the “second rescuer” could be the first rescuer switching from manual chest compressions to use of an ACD device.

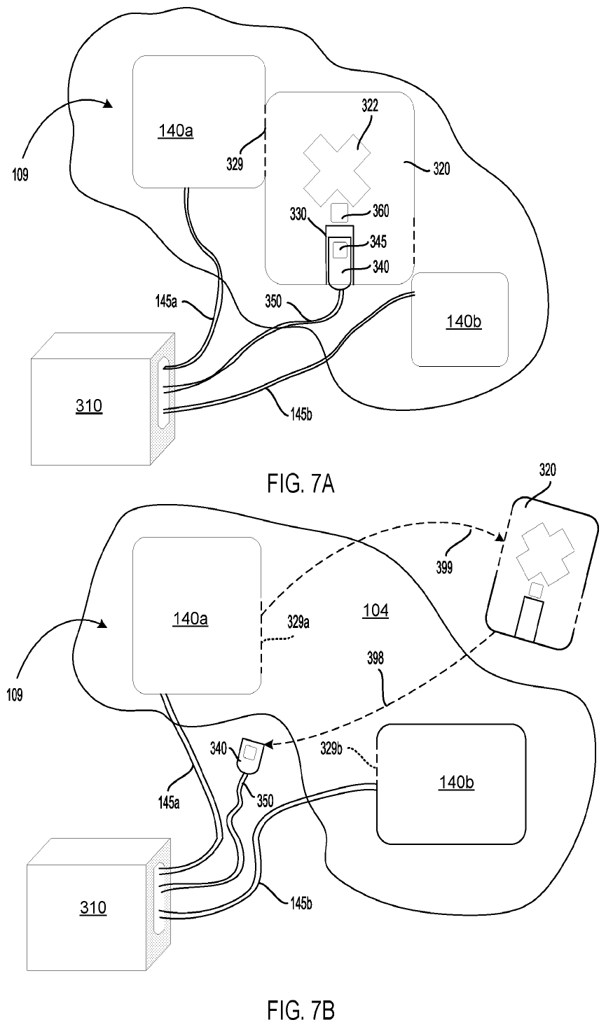

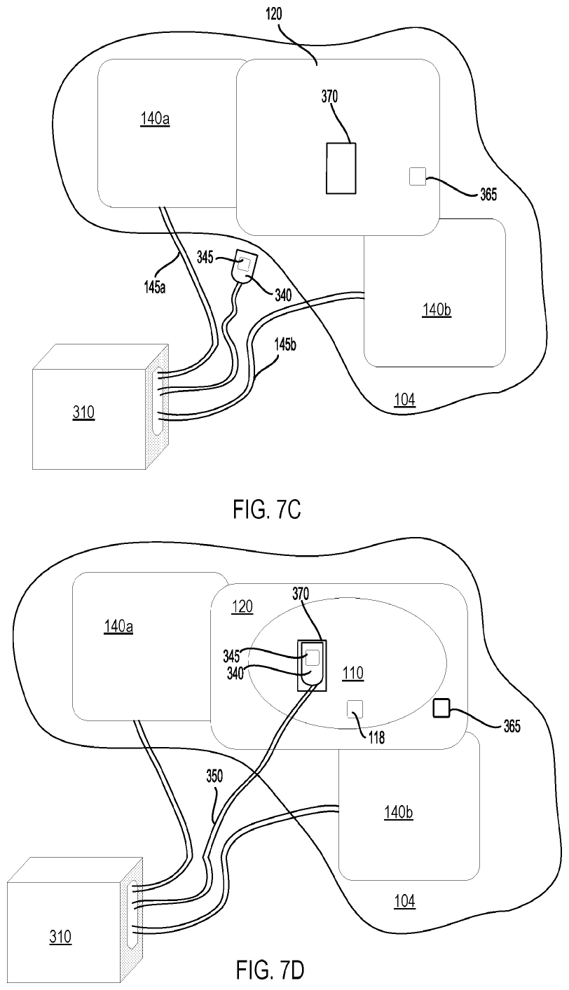

FIGS. 7A-7D are hybrid block and schematic diagrams illustrating the steps for transitioning from manual compressions to chest compressions with an ACD device 110.

Referring to FIG. 7A, an electrode assembly 109 is releasably coupled to the chest of the patient, for example, the electrode assembly 109 may be adhered to the patient's chest via an adhesive. In the illustrated example, the electrode assembly 109 includes at least the electrodes 140a and 140b and a manual compression pad 320. The electrodes 140a and 140b are electrically coupled to the patient monitor 310 via the high voltage cables 145a and 145b, respectively. The manual compression pad 320 additionally includes the one or more sensors 360. For example, the sensor(s) 360 may be embedded in the manual compression pad 320 and may include one or more accelerometers. In another implementation, the manual compression pad 320 may include a hand position indication 322, which indicates the proper position of the hands of the rescuer during standard manual chest compressions. This hand position indication 322 may be graphical and/or textual. The manual compression pad 320 may further include a connector receptacle 330, which is configured to accept a connector 340. The connector 340 includes a data communication circuit 345 (e.g., a first data communication circuit or a connector data communication circuit) configured to receive motion signals from the one or more sensors 360.

In an implementation, the data communication circuit 345 is configured to provide data indicative of the motion signals generated from the sensor(s) 360 to the patient monitor 310 via a cable 350. Several configurations for connecting the patient monitor with the electrodes and/or landing pads are illustrated in FIGS. 11A-11E. However, these example configurations are not limiting of the disclosure and in various implementations, the data communication circuit 345 is additionally, or alternatively, configured to communicate wirelessly with the patient monitor 310.

As shown in FIG. 7B, in preparation for a transition from manual chest compressions to compressions delivered via the ACD device 110, the rescuer 102 may remove the connector 340 from the compression pad connector receptacle 330 as indicated schematically by the arrow 398. However, the connector 340 may remain coupled to the patient monitor 310 via the cable 350. Additionally, in some examples, the electrode assembly 109 may optionally include a perforations 329a, 329b between the manual compression pad 320 and the electrodes 140a, 140b for easy removal. The rescuer 102 may separate the manual compression pad 320 from the electrode 140a along the perforations 329a, 329b, as indicated schematically by the arrow 399. During the separation of the manual compression pad 320 from the electrodes 140a, 140b, the electrodes 140a and 140b may remain in place and electrically connected to the patient monitor 310. In an embodiment, the rescuer 102 may separate the manual compression pad 320 from the electrode 140a using a cutting implement such as a scissors in addition to, or as an alternative to, using the perforations 329a, 329b.

Referring to FIG. 7C, following removal of the manual compression pad 320, the rescuer 102 may position the ACD device landing pad 120 on the chest 104 and releasably couple the ACD device landing pad 120 to the chest 104. The ACD device landing pad 120 may optionally include the ACD device mount 130. The ACD device landing pad 120 may further include one or more sensors 365 (e.g., acceleration sensors, force sensors, magnetic field sensors, gyroscopic sensors, proximity sensors, position sensors, rotation sensors, tilt sensors, orientation sensors, and gravity sensors) embedded in the ACD device landing pad 120. In an embodiment, the ACD device landing pad 120 includes a motion sensor (e.g., accelerometer), for measuring motion of the chest wall during ACD treatment. In another embodiment, the ACD device landing pad 120 includes a force sensor, for measuring force applied to the chest during ACD treatment. In another embodiment, the ACD device landing pad 120 includes a motion sensor (e.g., accelerometer) and a force sensor, for measuring motion of the chest wall and force applied to the chest during ACD treatment.

In embodiments, the ACD device landing pad 120 may overlap one or more of the electrodes 140a and 140b as shown for example in FIG. 7C. In an alternative implementation, the rescuer 102 may place the ACD device landing pad 120 on the chest 104 such that there is a space or gap between one or more of the electrodes 140a and 140b. In this case, the ACD device landing pad 120 may not overlap all of the electrodes 140a and 140b or may not overlap any of the electrodes 140a and 140b.

Referring to FIG. 7D, once the ACD device landing pad 120 is in place on the chest 104, the rescuer 102 may mechanically couple the ACD device 110 to the ACD device mount 130. The ACD device 110 may include a compression device connector receptacle 370. The connector receptacle 370 is configured to accept the connector 340. The rescuer 102 may couple the connector 340 to the connector receptacle 370. In FIG. 7D, the connector 340 is shown coupled to the connector receptacle 370. A processor 380 of the ACD device 110 is configured to receive motion signals 397 from the motion sensor 118 and further configured to provide data indicative of the motion signals 397 to the data communication circuit 345 of the connector 340. The data communication circuit 345 is configured to provide the data to the patient monitor 310 via the cable 350. The motion signals 397 are indicative of motion of the chest 104 during the ACD compressions administered via the ACD device 110.

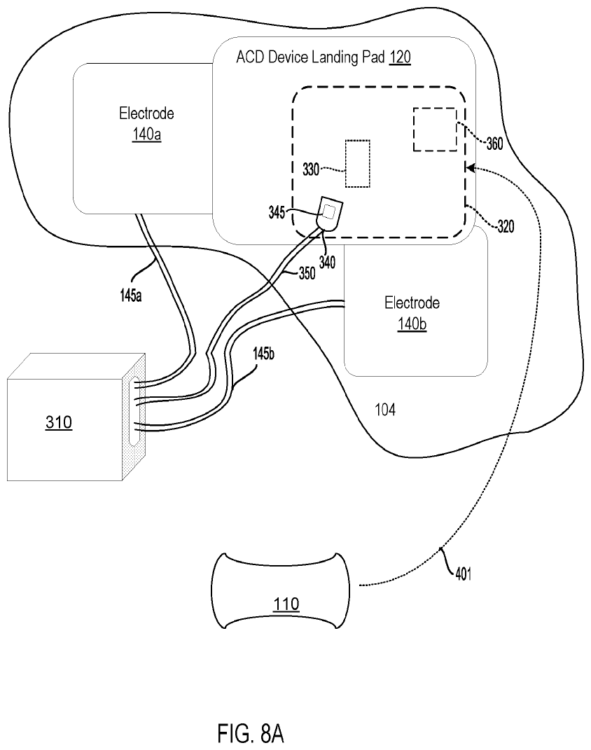

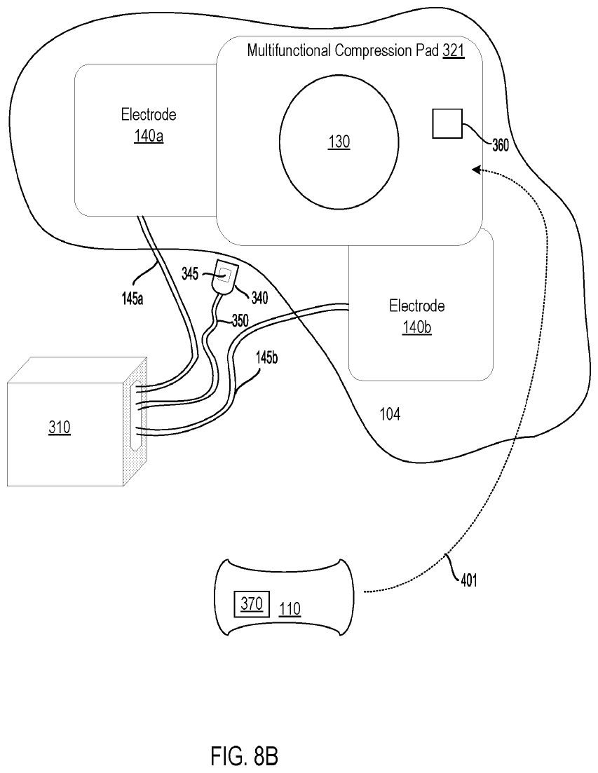

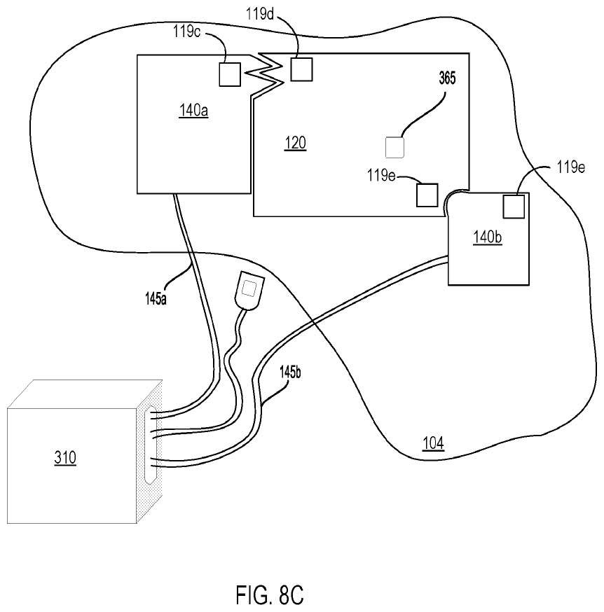

FIG. 8A is a hybrid block and schematic diagraming illustrating an alternative embodiment in which the landing pad 120 is placed on top of the manual compression pad 320. In this scenario, the defibrillator has arrived to the emergency scene prior to the ACD device, and so the electrodes and manual compression pad are first placed on the patient. The ACD device arrives subsequently thereafter. Rather than removing and replacing the manual compression pad 320, the rescuer 102 applies the landing pad 120 over the manual compression pad, and attaches the ACD device 110 to the landing pad 120. The benefit of this embodiment is that it reduces the number of steps that must completed by rescuers during a change from manual to ACD chest compressions.

In one embodiment, the cable 150 is disconnected and removed from the manual compression pad 320 and reconnected to the ACD landing pad or ACD device. This severs communication between the manual compression pad 320 and the patient monitor. Although, in some embodiments (as detailed in FIGS. 11A-11G) the manual compression pad may still wirelessly communicate with the patent monitor. In this embodiment, the ACD device 110 and/or patient monitor communicates (e.g., via proximity sensors, RFID, NFC, Wi-Fi, Bluetooth, etc.) with the manual compression pad during chest compressions.

In an alternative embodiment, the wired connection remains attached to the manual compression pad 320 when the landing pad 120 is placed on the manual pad. In this embodiment, the ACD device 110 then communicates with the patient monitor wirelessly (e.g., RFID, NFC, Wi-Fi, Bluetooth, etc.) or via a second cable (not shown) to the patient monitor 310. Because the manual compression pad is already directly connected to the patient monitor via the cable 150, the ACD device may acquire the signals from the landing pad via the proximity sensors, and then transmit the signals to the patient monitor, for further processing.

FIG. 8B illustrates an example of the multifunctional chest compression pad 321 in which the manual compression pad is also the ACD device landing pad. In this example, the ACD device 110 is affixed directly to the manual compression pad, which is equipped with sensors (e.g., motion sensor and/or force sensor) able to sense information from both manual chest compressions provided by the rescuers and chest compressions from the ACD device 110.

FIG. 8C illustrates an example of an ACD device landing pad 120 that includes patterns at two corners to ensure the correct orientation of the electrodes 140a, 140b with respect to the landing pad 120 and, hence, the patient's body. When the electrodes are properly placed, an electrical vector is formed through the heart for electrical therapy to be provided thereto. The purpose of patterned corners is to provide a highly recognizable orientation between the electrodes 140a, 140b and the landing pad 120 to ensure electrodes and the pad are properly situated on the chest of the patient. Similar patterns could also be implemented with the manual compression pad 320 or multifunctional chest compression pad. In certain embodiments, the electrodes 140a, 140b and landing pad 120 have proximity sensors 119c-119f (e.g., NFC tags, RFID, Bluetooth transmitters receivers) that allow for mutual data communication to occur between the defibrillator/monitor and the landing pad via the electrodes.

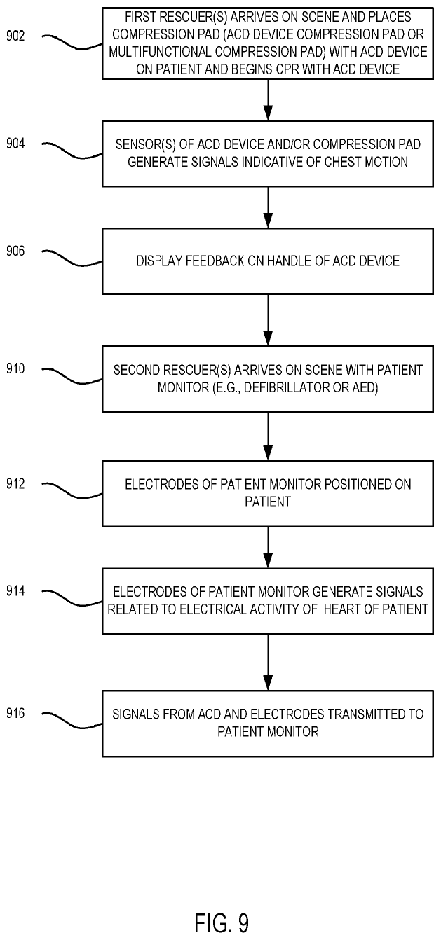

FIG. 9 refers to a scenario in which embodiments of the present disclosure may be employed. In a first step 902, the first rescuer arrives on scene and places a multifunctional chest compression pad 321 on the chest 104 of the patient 106 and begins performing CPR with the ACD device 110. The motion and/or force sensors of the ACD device and/or compression pad generate signals indicative of chest wall motion of the patient 106 and/or force applied to the patient in step 904. Then, in step 906, feedback is displayed on the handle 115 of the ACD device 110 (e.g., as detailed with respect to FIGS. 5A and/or 5B). As discussed above, the ACD device 110 may include a display that provides appropriate ACD feedback to the rescuer based on the sensed motion and/or force.

In the next step 910, the second rescuer arrives on scene with patient monitor 310 (e.g., a defibrillator or AED). The rescuer 102 connects the ACD device to the patient monitor 310 in order to transmit the signals generated by the sensors of the ACD device to the patient monitor (e.g., via cable 150). These generated signals may be filtered, analyzed, processor and/or combined with the signal from the multifunctional chest compression pad by the processor 380 of the ACD device 110 in optional step 908 (as detailed in previous embodiments). The rescuer positions the electrodes 140a, 140b of the patient monitor on the chest of the patient in step 912. In step 914, the electrodes 140a, 140b of the patient monitor 310 generate signals related to electrical activity of the heart (ECG data) of patient 106. Lastly, in step 916, the signals from the ACD device 110 and electrodes 140a, 140b are transmitted to the patient monitor 310.

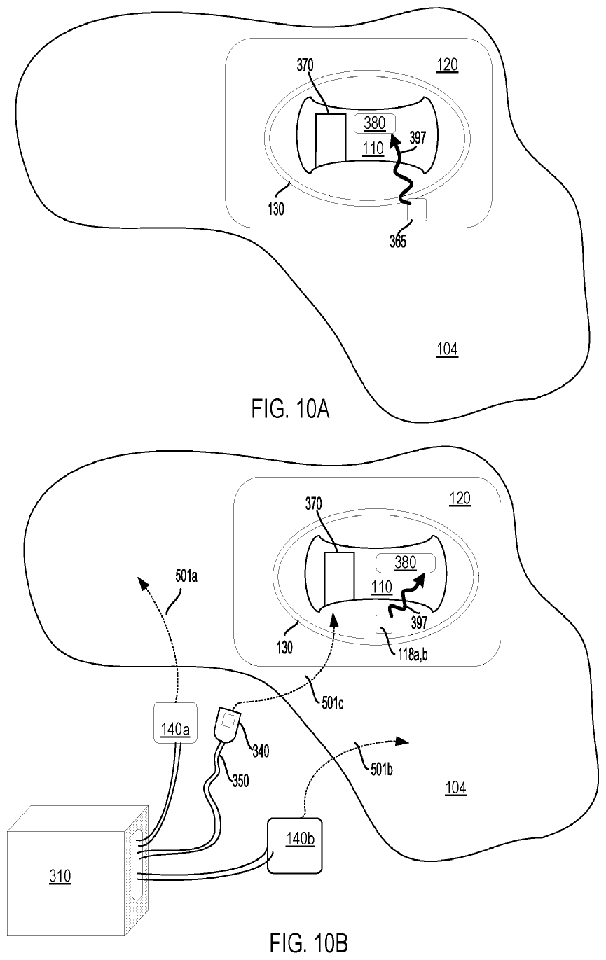

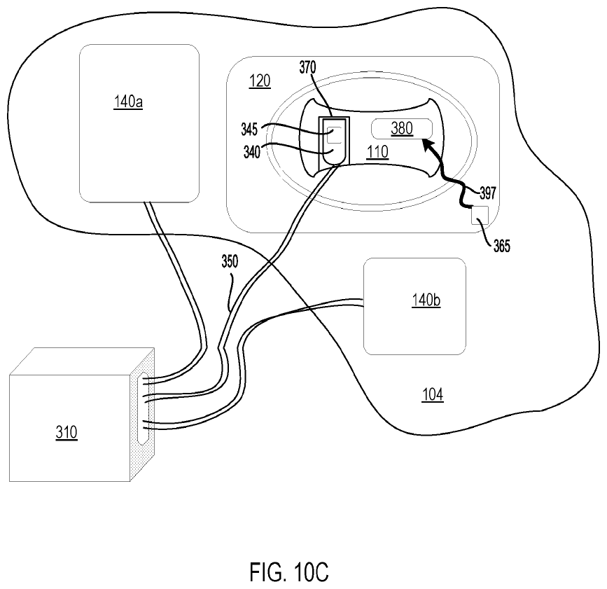

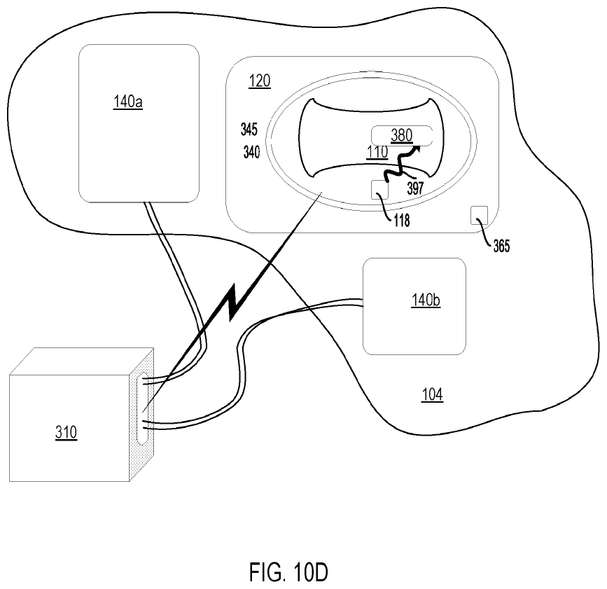

Referring to FIGS. 10A-10C, schematic diagrams of equipment configurations for a transition from ACD compressions (without electrodes and a patient monitor) to an integrated system that includes both an ACD device 110 and the patient monitor 310, with electrodes 140a, 140b, are shown.

Referring to FIG. 10A, the ACD device landing pad 120 is shown in position on the chest 104 prior to the addition of electrodes 140a, 140b of patient monitor 310. The ACD device 110 is shown in the ACD device mount 130 in this embodiment. During ACD chest compressions, the processor 380 of the ACD device may receive motion signals (indicated by arrow 397) from the one or more sensors 365 disposed in or on the landing pad 120. It should be appreciated that the sensor 365 is not required to be located in the position depicted in this figure, for example, the sensor 365 may be located at a position directly between the handle of the ACD device and the patient, for more precise sensing of chest wall motion.

In FIG. 10B, the patient monitor 310 with electrodes 140a, 140b arrives. This figure illustrates how the electrodes 140a, 140b of the patient monitor 310 and the connector 340 are affixed to the patient. The rescuer 102 may affix the electrodes 140a and 140b to the chest 104 as illustrated schematically by the arrows 501a and 501b. Further, the rescuer 102 may couple the connector 340 to the connector receptacle 370 as illustrated schematically by the arrow 501c. In FIG. 10C, the connector 340 is shown coupled to the connector receptacle 370 of the ACD device, and the electrodes 140a and 140b are shown affixed to the chest 104 of the patient. The processor 380 of the ACD device 110 is configured to receive the motion signals 397 from the sensor 365 and further configured to provide data indicative of the motion signals 397 to the data communication circuit 345 of the connector 340.

The above described procedures may offer various advantages. For example, during positioning and coupling of the ACD device landing pad 120 on the chest 104, the electrodes 140a and 140b may remain coupled to the chest 104 and coupled to the patient monitor 310 via the high voltage cables 145a and 145b. As such, this procedure may eliminate the need to rearrange and/or disconnect and reconnect the electrodes 140a and 140b. Thus, this procedure may avoid any delays, interruptions, and/or missteps in care that may result from these rearrangements and/or disconnections and reconnections.

FIG. 10D is an alternative embodiment in which communication between the ACD device 110 and the patient monitor 310 is wireless (e.g., Bluetooth, NFC, RFID, 802.11, cellular, etc.).

In a typical implementation, the patient monitor and ACD device have been previously paired (e.g., by an employer or by the manufacturer) such that upon activation, the devices initially search for a device to pair with. After initialization, the devices will periodically search for devices to connect with. Alternatively, the ACD device and patient monitor may each include an input (e.g., button), which cause the ACD device or patient monitor to search for compatible devices. Thus, anytime a new wireless device or component need to be paired with the ACD device or patient monitor, the rescuer simply presses the input.

Depending on the quality of the connection and a measured bitrate, the ACD device or patient monitor may compress the data signal to reduce the amount of data that must be transferred.

FIGS. 11A-11E are block diagrams illustrating several different possible configurations of the components of the integrated ACD device 110 and patient monitor 310 and how those devices may communicate with one another.

Referring to FIG. 11A, the manual compression pad 320 is affixed to the chest of the patient. Disposed on the manual compression pad 320 is the connector receptacle 330, which interfaces with the connector 340 of the patient monitor 310. A motion sensor 360 (e.g., accelerometer) of the manual compression pad measures chest compression motion and transmits the signal information to the connector receptacle 330. The connector 340 includes data communication circuit 345, which is configured to communicate with the patient monitor 310 via the cable 350. More specifically, the cable 350 connects with the data communication circuit 611 of the patient monitor 310, which is further connected to the processor 621 and memory 631 of the patient monitor 310.

FIG. 11B is similar to FIG. 11A, however, in this embodiment, the data communication circuit 611 of the patient monitor connects wirelessly with the connector receptacle 330 of the manual compression pad 320. In this embodiment, the connector receptacle 330 includes circuitry for wireless communication (e.g., Bluetooth, NFC, RFID, 802.11, cellular, etc.).

Referring to FIG. 11C, the ACD device landing pad 120 is affixed to the chest of the patient. Additionally, the ACD device 110 is affixed to the landing pad 120. Disposed on ACD device 110 is the connector receptacle 370, which is configured to interface with the connector 340 of the patient monitor 310, to establish data communications there between. A motion sensor 365 (e.g., accelerometer) of the multifunctional landing pad 321 measures chest compression motion and transmits the signal information to the processor(s) 380 of the ACD device, which may or may not process the received signals. The processor(s) 380 transmits the signal to the connector receptacle 370, for further transmission to the patient monitor 310. As detailed before, the connector 340 includes the data communication circuit 345, which is configured to communicate to the patient monitor 310 via the cable 350.

FIG. 11D is similar to FIG. 11A, however, in this embodiment, the data communication circuit 611 of the patient monitor 310 connects wirelessly with the connector receptacle 370 of the landing pad 120. In this embodiment, the connector receptacle 330 includes circuitry for wireless communication (e.g., Bluetooth, NFC, RFID, 802.11, cellular, etc.).

FIG. 11E illustrates yet another alternative embodiment. In this example the ACD device 110 communicates wirelessly with the electrode pads of the patient monitor 310. This communication is illustrated by communication pathways 799a, 799b. In this embodiment, communication circuitry (e.g., NFC tags, Bluetooth transmitters/receivers illustrated in FIG. 8C) is included in both the connector receptacle 370 and electrode pads 140a, 140b to enable wireless communication between ACD device and patient monitor. A benefit of this configuration is that the rescuer(s) would not need to connect and/or disconnect the cable 150. Rather, when positioned in close enough proximity, a wireless communication connection may be established between the ACD device and patient monitor, via the electrodes 140a, 140b.

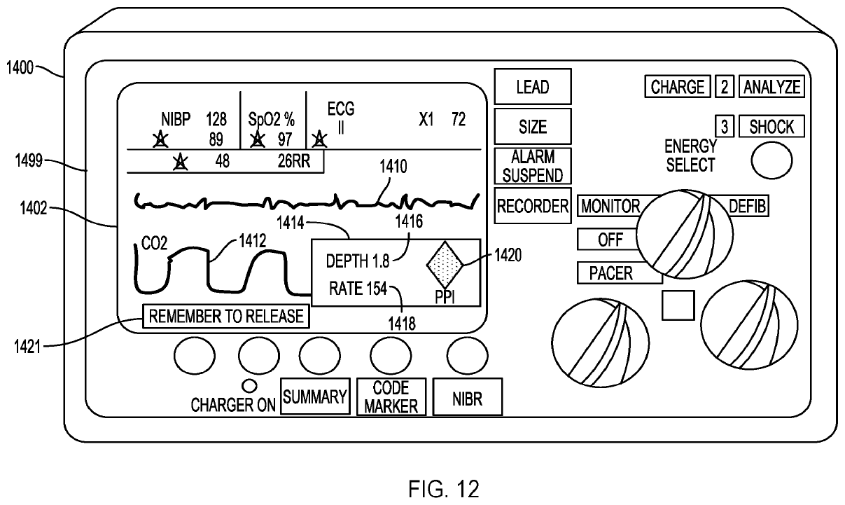

FIG. 12 is a schematic diagram of an example of a patient monitor taking the form of a professional style defibrillator 1400 configured to provide real-time feedback to the rescuer 102. In the illustrated embodiment, according to an implementation, the computing device 160 may be the defibrillator 1400. The defibrillator 1400 may include a dashboard 1499, which further includes a display 1402.

The display may include an ECG waveform 1410 by gathering ECG data points and sensor readings and processing motion-induced (e.g., CPR-induced) noise out of the ECG waveform. As an example of a defibrillator dashboard layout, the ECG waveform 1410 may be a full-length waveform that may fill the entire span of the display device, while the second waveform (e.g., the CO2 waveform 1412) may be a partial-length waveform that fills only a portion of the display. A portion of the display beside the second waveform provides the CPR information in box 1414. For example, the display may split the horizontal area for the second waveform in half, displaying waveform 1412 on left, and CPR information on the right in box 1414. However, the layout, configuration, and included information for the dashboard 1499 as described above are examples only and other layouts, configurations, and included information are within the scope of the disclosure.

The CPR display parameters related to the performance of CPR and these parameters may be displayed automatically in response to detecting chest compressions (e.g., by sensors of landing pads or ACD device). For example, the CPR parameters may include the chest compression rate 1418 (e.g., number of compression cycles per minute) and the chest compression depth 1416 (e.g., depth of compressions in inches or millimeters). Displaying the measured rate and depth data, in addition to, or instead of, an indication of whether the values are within or outside of an acceptable range may enhance the value of the feedback for the rescuer. For example, if an acceptable range for chest compression depth is 25 to 60 mm, providing the rescuer with an indication that his/her compressions and decompressions are only 15 mm may allow the rescuer to determine how to correctly modify his/her administration of the chest compressions and decompressions (e.g., he or she can know how much to increase effort, and not merely that effort should be increased some unknown amount).

The CPR dashboard 1414 may also include a perfusion performance indicator (PPI) 1420. The PPI 1420 may be a geometric shape (e.g., a diamond, square, a rectangle, a circle, a triangle, or other polygon) with an amount of fill that is in the shape differing over time to provide feedback about one or more of the rate and depth of the chest compressions. When the rescuer performs manual CPR adequately (e.g., according to ACLS guidelines and/or at a rate of about 100 compressions and decompressions per minute (CPM) with the depth of each compression greater than 40 mm) the fill will cover the entire area of the geometric shape (e.g., the entire indicator may be filled). As the rate and/or depth decreases below acceptable limits, the fraction of the filled area of the geometric shape decreases. The PPI 1420 may provide a visual indication of the quality of the CPR. Further, the PPI 1420 may provide a target for the rescuer to keep the PPI 1420 completely filled.

As another feedback example, a reminder 1421 regarding “release” in performing chest compression. Specifically, a fatigued rescuer may lean forward on the chest of a patient and not sufficiently release pressure on the sternum of the patient at the top of each decompression stroke. This may reduce the perfusion and circulation accomplished by the chest compressions. The dashboard 1499 may provide the release reminder 1421 when the defibrillator processor 621 determines that the rescuer is not sufficiently releasing. For example, signals from the sensors 118a, 188a, 360,365 may exhibit an “end” to the compression cycle that is flat and thus indicates that the rescuer is maintaining pressure on the sternum to an unnecessary degree.

In an implementation, when a patient case is initiated, the feedback mode configuration setting may initialize at the default setting. If the feedback mode configuration setting changes the selected feedback mode for a first case, then when a second case begins, the configuration setting may automatically revert back to the default setting. The control software may recognize and/or identify initiation of the patient case based on one or more events.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device.

A computer program is a set of instructions that can be used, directly or indirectly, in a computer to perform some activity or bring about some result. A computer program can be written in any form of programming language, including compiled or interpreted languages, and it can be deployed in any form, including as a stand-alone program or as a module, component, subroutine, or other unit suitable for use in a computing environment. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks.

The computing device 160 described herein may include, or be operatively coupled to communicate with, one or more mass storage devices for storing data files; such devices include magnetic disks, such as internal hard disks and removable disks; magneto-optical disks; and optical disks.

The terms “machine-readable medium,” “computer-readable medium,” and “processor-readable medium” as used herein, refer to any medium that participates in providing data that causes a machine to operate in a specific fashion. Using a computer system, various processor-readable media (e.g., a computer program product) might be involved in providing instructions/code to processor(s) for execution and/or might be used to store and/or carry such instructions/code (e.g., as signals).

In many implementations, a processor-readable medium is a physical and/or tangible storage medium. Such a medium may take many forms, including but not limited to, non-volatile media and volatile media. Non-volatile media include, for example, optical and/or magnetic disks. Volatile media include, without limitation, dynamic memory.

Common forms of physical and/or tangible processor-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, EPROM, a FLASH-EPROM, any other memory chip or cartridge, a carrier wave as described hereinafter, or any other medium from which a computer can read instructions and/or code.

Various forms of processor-readable media may be involved in carrying one or more sequences of one or more instructions to one or more processors for execution. Merely by way of example, the instructions may initially be carried on a flash device, a device including persistent memory, and/or a magnetic disk and/or optical disc of a remote computer. A remote computer might load the instructions into its dynamic memory and send the instructions as signals over a transmission medium to be received and/or executed by a computer system.

The computing device 160 may be part of a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet. The computer system can include clients and servers. A client and server are generally remote from each other and typically interact through a network, such as the described one. The relationship of client and server arises by virtue of computer programs running on the respective computers and having a client-server relationship to each other.

Substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

Information and signals may be represented using any of a variety of different technologies and techniques. For example, data, instructions, commands, information, signals, and symbols that may be referenced throughout the above description may be represented by voltages, currents, electromagnetic waves, magnetic fields or particles, optical fields or particles, or any combination thereof.

The methods, systems, and devices discussed above are examples. Various alternative configurations may omit, substitute, or add various procedures or components as appropriate. Configurations may be described as a process which is depicted as a flow diagram or block diagram. Although each may describe the operations as a sequential process, many of the operations can be performed in parallel or concurrently. In addition, the order of the operations may be rearranged. A process may have additional stages not included in the figure. Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the scope of the disclosure.

Also, configurations may be described as a process which is depicted as a flow diagram or block diagram. Although each may describe the operations as a sequential process, many of the operations can be performed in parallel or concurrently. In addition, the order of the operations may be rearranged. A process may have additional stages or functions not included in the figure. Furthermore, examples of the methods may be implemented by hardware, software, firmware, middleware, microcode, hardware description languages, or any combination thereof. When implemented in software, firmware, middleware, or microcode, the program code or code segments to perform the tasks may be stored in a non-transitory processor-readable medium such as a storage medium. Processors may perform the described tasks.

Components, functional or otherwise, shown in the figures and/or discussed herein as being connected or communicating with each other are communicatively coupled. That is, they may be directly or indirectly connected to enable communication between them.

As used herein, including in the claims, “and” as used in a list of items prefaced by “at least one of” indicates a disjunctive list such that, for example, a list of “at least one of A, B, and C” means A or B or C or AB or AC or BC or ABC (i.e., A and B and C), or combinations with more than one feature (e.g., AA, AAB, ABBC, etc.). As used herein, including in the claims, unless otherwise stated, a statement that a function or operation is “based on” an item or condition means that the function or operation is based on the stated item or condition and may be based on one or more items and/or conditions in addition to the stated item or condition.

Having described several example configurations, various modifications, alternative constructions, and equivalents may be used without departing from the disclosure. For example, the above elements may be components of a larger system, wherein other rules may take precedence over or otherwise modify the application of aspects of the present disclosure. Also, a number of operations may be undertaken before, during, or after the above elements are considered. Also, technology evolves and, thus, many of the elements are examples and do not bound the scope of the disclosure or claims. Accordingly, the above description does not bound the scope of the claims.

Other embodiments are within the scope of the present disclosure. For example, due to the nature of software, functions described above can be implemented using software, hardware, firmware, hardwiring, or combinations of any of these. Features implementing functions may also be physically located at various locations, including being distributed such that portions of functions are implemented at different physical locations.

1. A system for providing resuscitative therapy to a patient by delivering active chest compression decompressions, comprising:
an ACD device configured to be coupled to the patient's chest and constructed for administering of active compression decompression therapy, the ACD device comprising:
at least one sensor configured for sensing at least one active compression decompression parameter,
processing circuitry configured to process the at least one active compression decompression parameter and generate output based at least in part on the at least one parameter, and
a first communication circuit configured for use in transferring data related to the output from the ACD device to a patient monitor; and
a second communication circuit, configured for use in the transferring of the data related to the output from the ACD device to the patient monitor, capable of being removably coupled to at least one of the ACD device and the patient monitor.

2. The system of claim 1, further comprising a connector, comprising the second communication circuit, for use in establishing communication from the ACD device to the patient monitor.

3. The system of claim 1, wherein the at least one sensor comprises at least one of a motion sensor, an accelerometer, and a force sensor, wherein the at least one of the motion sensor, the accelerometer, and the force sensor is configured for monitoring at least one of: motion of at least a portion of the patient's chest in response to force applied during the active compression decompression therapy, and force applied to at least a portion of the patient's chest during the active compression decompression therapy.

4-8. (canceled)

9. The system of claim 1, wherein the ACD device includes a pad configured to be adhered to and to cover at least a portion of the patient's chest.

10-12. (canceled)

13. The system of claim 9, wherein the pad includes at least one receptacle for receiving an electrode assembly of the patient monitor.

14-15. (canceled)

16. The system of claim 1, wherein the patient monitor comprises an electrode assembly, and wherein the electrode assembly includes a first electrode and a second electrode electrically coupled to one another and each configured to be adhered to the patient to deliver electrical therapy to the patient.

17. (canceled)

18. The system of claim 1, wherein the patient monitor comprises a defibrillator.

19. The system of claim 16, wherein the electrode assembly includes a sensor configured to provide data related to chest compressions.

20. The system of claim 1, wherein the second communication circuit is configured to transfer data related to chest compressions.

21-25. (canceled)

26. The system of claim 1, wherein the first and second communication circuits are configured to mutually establish a wireless communications channel.

27-118. (canceled)

119. A system for providing resuscitative therapy to a patient by delivering active chest compression decompressions, comprising:
an active compression decompression (ACD) device configured to be positioned on a patient's chest and configured for administering of active compression decompression therapy, the ACD device comprising:
at least one sensor configured to sense at least one active compression decompression parameter, and
at least one processor and at least one memory, the at least one processor being configured to process the at least one parameter and generate an output based at least in part on the at least one parameter;
wherein the ACD device is configured such that:
while the ACD device is positioned on the patient's chest, at least one electrode, connected to or part of a defibrillator, can be positioned on the patient, to enable delivery of electrotherapy, without removing the ACD device from the patient's chest, and
while the ACD device is positioned on the patient's chest, when the defibrillator comes within sufficient proximity with the ACD device, the ACD device and the defibrillator wirelessly couple such that the output can be wirelessly transmitted from the ACD device to the defibrillator.

120. The system of claim 119, comprising the defibrillator, wherein the ACD device comprises a first communication circuit and the defibrillator comprises a second communication circuit, and wherein the first communication circuit and the second communication circuit are configured for use in enabling the ACD device and the defibrillator to wirelessly couple.

121. The system of claim 120, comprising a patient monitor, wherein the patient monitor comprises the defibrillator.

122. The system of claim 121, wherein the system is configured such that the at least one electrode can be positioned on the patient, to enable delivery of electrotherapy, without requiring removal of the ACD device from the patient's chest.

123. The system of claim 122, wherein the ACD device comprises a landing pad.

124. The system of claim 122, wherein the ACD device is configured to couple with a landing pad positioned on the patient's chest.

125. A system for providing resuscitative therapy to a patient by delivering active chest compression decompressions, comprising:
an active compression decompression (ACD) device configured to be positioned on a patient's chest and configured for administering of active compression decompression therapy, the ACD device comprising:
at least one sensor configured to sense at least one active compression decompression parameter, and
at least one processor and at least one memory, the at least one processor being configured to process the at least one parameter and generate an output based at least in part on the at least one parameter;
wherein the ACD device is configured such that:
while at least one electrode of a defibrillator is positioned on the patient, to enable delivery of electrotherapy, the ACD device can be positioned on the chest of the patient without removing the at least one electrode from the patient, and
while the at least one electrode is positioned on the patient, to enable delivery of electrotherapy, when the ACD device comes within sufficient proximity with the defibrillator, the defibrillator and the ACD device wirelessly couple such that the output can be wirelessly transmitted from the ACD device to the defibrillator.

126. The system of claim 125, comprising the defibrillator, wherein the ACD device comprises a first communication circuit and the defibrillator comprises a second communication circuit, and wherein the first communication circuit and the second communication circuit are configured for use in enabling the ACD device and the defibrillator to wirelessly couple.

127. The system of claim 126, comprising a patient monitor, wherein the patient monitor the defibrillator.

128. The system of claim 127, wherein the system is configured such that the ACD device can be positioned on the patient's chest, to enable delivery of active chest compression decompressions, without requiring removal of the at least one electrode from the patient.

129. The system of claim 128, wherein the ACD device comprises a landing pad.

130. The system of claim 128, wherein the ACD device is configured to couple with a landing pad positioned on the patient's chest.

131-139. (canceled)

140. The system of claim 1, wherein the ACD device is configured to pull up on the patient's chest.

141. The system of claim 119, wherein the ACD device is configured to pull up on the patient's chest.

142. The system of claim 125, wherein the ACD device is configured to pull up on the patient's chest.
